(19) United States
(12) Patent Application Publication
(10) Pub. No.: US 2022/0133279 A1
(43) Pub. Date: May 5, 2022

(54) METHOD AND LOCAL AND REGIONAL CLOUD INFRASTRUCTURE SYSTEM FOR PRESSURE ELASTOGRAPHY MEASUREMENT DEVICES

(71) Applicant: Sure, Inc., Palos Verdes Estates, CA (US)

(72) Inventors: Geoff Bricker, Holly Ridge, NC (US); David Ables, Chappaqua, NY (US); Joseph Peterson, Palos Verdes Estates, CA (US)

(21) Appl. No.: 17/516,942

(22) Filed: Nov. 2, 2021

Related U.S. Application Data
(60) Provisional application No. 63/108,522, filed on Nov. 2, 2020.

(51) Int. Cl. A61B 8/08; G06T 7/00; G06N 20/00; A61B 8/00; A61B 5/00

(52) U.S. Cl. CPC A61B 8/485; G06T 7/0012; A61B 8/0825; G06N 20/00; G06T 2207/10132; A61B 8/58; A61B 8/565; A61B 5/0022; G06T 2207/30068; A61B 8/5223

(57) ABSTRACT

An exemplary method and system for a local cloud infrastructure are disclosed for a pressure elastography-based measurement system, e.g., to provide pre-screening/early screening for breast cancer detection and/or mass detection. The exemplary system comprises a local appliance and gateway that provides cloud infrastructure capabilities in a portable manner to be deployable in a doctor's office or clinic. The exemplary system can operate independently, as well as in conjunction with a regional or global cloud infrastructure, to provide electronic medical record capabilities, appointment management capabilities, as well as teleradiology interface capabilities, e.g., to improve examination workflow and reduce overall operation cost.

### RELATED APPLICATION

This application claims priority to, and the benefit of, U.S. Provisional Patent Application No. 63/108,522, filed Nov. 2, 2020, entitled “Method and Local and Cloud Infrastructure System for Pressure Elastography Measurement Devices,” which is incorporated by reference herein in its entirety.

### BACKGROUND

There is a large U.S. and global population of women who do not participate in clinician-assisted breast cancer examination or screening. Many of these women are in a “detection gap” in part due to the expense and nature associated with mammography. In the United States, approximately 40 million women do not receive annual breast cancer examinations. There is a need to address such a detection gap among women having both normal and increased risk for breast cancer who fall outside or between guidelines-related mammography who would benefit from regular early detection measures. The need is substantially higher in countries with developing healthcare systems.

Pressure elastography devices have been clinically available as an alternative to mammography that can provide, in a small hand-held device, a high-resolution form of elastography measurement that can be universally adopted for the first-line locus of breast mass detection. In addition, portable ultrasound technology has become more prevalent but is still not generally used in early detection examinations due to its associated expense.

Nevertheless, improvements can be made to breast cancer examination or screening procedures to make them more cost-effective, to improve acquisition quality, and to improve their workflow. For healthcare systems with developing healthcare infrastructure, there is a benefit to providing similar capabilities in telemedicine and teleradiology that are readily available in more developed healthcare infrastructure.

### SUMMARY

An exemplary method and system for a local cloud infrastructure are disclosed for a pressure elastography-based measurement system, e.g., to provide pre-screening/early screening for breast cancer detection and/or mass detection. The exemplary method and system may include ultrasound sensors for ultrasonic measurements, e.g., for intermediate screening or validation. The exemplary system comprises a local appliance and gateway that provides cloud infrastructure capabilities in a portable manner to be deployable in a doctor's office or clinic. The exemplary system can operate independently, as well as in conjunction with a regional or global cloud infrastructure, to provide electronic medical record capabilities, appointment management capabilities, as well as teleradiology interface capabilities, e.g., to improve examination workflow and reduce overall operation cost. Notably, the exemplary system comprising the local appliance can be configured with the same, or substantially similar, implementation as the regional or global cloud infrastructure. This doppelganger or twin capabilities provide for a seamless and unified operation that allows clinicians to interchangeably access clinical data, patient information, and other information between the local cloud infrastructure of the appliance and the regional or global cloud infrastructure. In addition, upgrades or modifications to the local appliance and gateway can be readily implemented in the doppelganger or twin regional or cloud infrastructure, reducing the cost of system maintenance and operation.

The exemplary system can be configured with logging, monitoring, and/or assessment capabilities, e.g., for scan quality, examiner quality, among others, including business metrics. The infrastructure of the exemplary method and system can be readily expanded to provide advanced versions of such capabilities via algorithms, e.g., machine learning and artificial intelligence.

In conjunction with the regional or global cloud infrastructure, the exemplary system comprising the local appliance and gateway (e.g., having the electronic medical record capabilities, appointment management capabilities, as well as teleradiology interface capabilities) can be readily deployed in a secure manner compliant with healthcare privacy rules, e.g., relating to patient data, such as with the Health Insurance Portability and Accountability Act (HIPAA), the European Union General Data Protection Regulation, and various laws and regulations of specific countries.

A “clinician” as used herein refers to any health professional, licensed or otherwise, that is trained and/or certified to conduct pre-screening or early screening of breast cancer detection and/or mass detection using pressure elastography measurements.

In an aspect, a method (e.g., for a local appliance and gateway, e.g., SureSync Appliance) is disclosed comprising receiving, by a processor, at a data collection appliance and gateway, a first data set associated with a pressure elastography measurement (e.g., tactile imaging, palpation imaging, mechanical imaging)) of a breast mass detection procedure conducted on a subject (e.g., acquired from a hand-held device comprising a capacitive sensor array); receiving, by the processor, at the data collection appliance and gateway, a second data set associated with an ultrasound scan of a second breast mass detection procedure contemporaneously conducted on the subject following the pressure elastography measurement (e.g., acquired from a portable ultrasound device); storing, by the processor, at the data collection appliance and gateway, the first data set and the second data set; and transmitting, by the processor of the data collection appliance and gateway, the first data set and the second data set to a remote or cloud server, wherein the first data set and the second data set are (i) subsequently presented on a display of a computing device or in a report for use in an assessment of a breast mass and/or (ii) subsequently analyzed via machine learning or deep learning operations (e.g., in a remote/cloud server or the device) to provide an indication associated with the assessment of the breast mass.

In some embodiments, the method includes analyzing, by a processor of the remote or cloud server or the data collection appliance and gateway, the first data set (associated with the pressure elastography measurement) to assess metrics associated with the quality of acquisition of the pressure elastography measurement (e.g., using pressure & ultrasound data in conjunction and in contrast with each other to assess the quality of each).

In some embodiments, the metrics associated with quality of acquisition comprises at least one of the metrics associated with examiner monitoring, metrics associated with workflow monitoring, and metrics associated with device monitoring.

In some embodiments, the metrics associated with examiner monitoring comprise any one of a log of time spent per breast during an exam; a log of percent time outside pre-defined force region; a log of a number of recording deleted; a log of a number of re-recording; log of total time spent per breast and per patient during an exam; a log of an average force used during a different portion of the exam; a log of variations from recommendations and deviations from pre-defined ranges of operations (e.g., too long/short per patient, an abnormal ratio of masses on left v. right breast, using a wrong amount of exam force).

In some embodiments, the metrics associated with workflow monitoring comprise a log of significant deviations from training that may indicate a need to change recommendations (e.g., if a majority of examiners are consistently over or under recommended force, then re-evaluate recommendations; time per patient/per breast changes with experience to guide training).

In some embodiments, the metrics associated with device monitoring comprises at least one of hardware status (e.g., device status or tablet status); a log of hardware status indicative of wear (e.g., battery levels over the course of a day); and a log of daily calibration data to inspect sensor data to look for problems, e.g., broken electrical connection or possible delamination of sensor layers; tracking v. sensor serial number allowing the identifying quality issues with particular lots or assembly houses.

In some embodiments, the method further includes acquiring, by the processor, a third data set associated with the subject, the breast mass detection procedure, and the second breast mass detection procedure; and transmitting, by the processor, the third data set to the remote or cloud server, wherein the third data set are used with the first data set and the second data set to be (i) subsequently presented on the display of the computing device or in the report for use in the assessment of the breast mass or ii) subsequently analyzed via the machine learning or deep learning operations to provide the indication associated with the assessment of the breast mass.

In some embodiments, the first data set is acquired by authenticating, by a processor (e.g., of an acquisition device), an examiner credential comprising an examiner identifier; retrieving, by the processor (e.g., of an acquisition device), from a remote or cloud database, through the data collection appliance and gateway, a data set comprising a list of clinic or center (e.g., certified or eligible clinic or center); and presenting, by the processor (e.g., of the acquisition device), at a user interface (e.g., of the acquisition device), the list of clinic or center, wherein the examiner identifier is associated with at least the pressure elastography measurement and used for quality monitoring associated with the examiner identifier (or accepting an identifier associated with a clinic or center and examiner identifier).

In some embodiments, the method further includes presenting, through a teleradiology interface (e.g., of the acquisition device), focused ultrasounds of detected masses while exam in-process.

In some embodiments, the method further includes transmitting, by the processor (e.g., of the remote or cloud server), a notification of a potential mass to an on-call radiologist for review upon the identification of the mass by an automated analysis system.

In some embodiments, the method further includes generating, by processor (e.g., of the remote or cloud server), a report of the pressure elastography measurement; and transmitting, by the processor (e.g., of the remote or cloud server), the report to a radiologist or pre-defined reviewer for review (e.g., wherein the method further include receiving radiologic results and including them in the report at the end of the exam).

In another aspect, a method (e.g., for an analytical engine or analysis system) is disclosed comprising obtaining, by a processor, a first data set associated with a pressure elastography measurement acquired during a breast mass detection procedure conducted on a subject; analyzing, by the processor, the first data set to determine one or more metrics associated with quality of acquisition, wherein the one or more metrics includes at least one of metrics associated with examiner monitoring, metrics associated with workflow monitoring, and metrics associated with device monitoring; and causing, by the processor, the one or more metrics to be outputted at a display or a report (e.g., for quality assessment of an examiner, a given device, a given workflow).

In some embodiments, the metrics associated with examiner monitoring comprise any one of a log of events; a log of applied force applied during the exam (e.g., 1 Hz, 10 Hz sampling, or therebetween); a log summary data throughout a given exam (e.g., 1 Hz, 10 Hz sampling, or therebetween); a log of time spent per breast during an exam; a log of percent time outside a pre-defined force region (e.g., wherein the exam comprises an assessment of at least four regions for each breast); a log of a number of recording deleted; a log of a number of re-recording; a log of total time spent per breast and per patient during an exam; a log of an average force used during a different portion of the exam; and a log of variations from recommendations and deviations from pre-defined ranges of operations (e.g., too long/short per patient, an abnormal ratio of masses on left v. right breast, using the wrong amount of exam force).

In some embodiments, the metrics associated with workflow monitoring comprise a log of significant deviations from training that may indicate a need to change recommendations (e.g., if the majority of examiners are consistently over or under recommended force, then re-evaluate recommendations; time per patient/per breast changes with experience to guide training).

In some embodiments, the metrics associated with device monitoring comprises at least one of hardware status (e.g., device status or tablet status); a log of hardware status indicative of wear (e.g., battery levels over the course of the day); and a log of daily calibration data to inspect sensor data to look for problems, e.g., broken electrical connection or possible delamination of sensor layers; tracking v. sensor serial number allowing the identifying quality issues with particular lots or assembly houses.

In some embodiments, the method further includes transmitting, by the processor, a notification of a potential mass to an on-call radiologist for review upon the identification of the mass by an analysis system.

In some embodiments, the method further includes generating, by a processor, a report of the pressure elastography measurement; and transmitting, by the processor, the report to a radiologist or pre-defined reviewer for review.

In another aspect, a method (e.g., for an acquisition device) is disclosed comprising acquiring, by a processor, via an acquisition device comprising a capacitive sensor array, a first data set associated with a pressure elastography measurement (e.g., tactile imaging, palpation imaging, mechanical imaging) of a breast mass detection procedure conducted on a subject; determining, by the processor, estimated size and estimated relative hardness of a detected mass; and transmitting, by the processor, the first data set and estimated size and estimated relative hardness of the detected mass to a remote or cloud server, wherein the first data set is (i) subsequently presented on a display of a computing device or in a report for use in an assessment of a breast mass or (ii) subsequently analyzed via machine learning or deep learning operations to provide an indication associated with the assessment of the breast mass.

In some embodiments, the method further includes authenticating, by the processor, an examiner credential comprising an examiner identifier; retrieving, by the processor, from the data collection appliance and gateway (e.g., from a remote or cloud database), through a data collection appliance and gateway, a data set comprising a list of clinic or center (e.g., certified or eligible clinic or center); and presenting, by the processor, at a user interface, the list of clinic or center, wherein the examiner identifier is associated with at least the pressure elastography measurement and used for quality monitoring associated with the examiner identifier (or accepting an identifier associated with a clinic or center and examiner identifier).

In some embodiments, the method further includes acquiring, by the processor, via the acquisition device comprising associated with an ultrasound scan of a second breast mass detection procedure contemporaneously conducted on the subject following the pressure elastography measurement (e.g., acquired from a portable ultrasound device); and transmitting, by the processor, the second data set to the remote or cloud server, wherein the first data set and second data set are (i) subsequently presented on the display of the computing device or in the report for use in the assessment of the breast mass or (ii) subsequently analyzed via the machine learning or deep learning operations to provide the indication associated with the assessment of the breast mass.

In some embodiments, the method further includes presenting, through a teleradiology interface, focused ultrasounds of detected masses while exam in-process.

In some embodiments, the first data set (e.g., associated with the pressure elastography measurement) comprises, for each detected mass, 5-10 seconds of acquired sensor measurement of the detected mass and an associated location of the detected mass.

In some embodiments, the first data set (e.g., associated with the pressure elastography measurement) further comprises, for each detected mass, estimated size and estimated relative hardness of the detected mass.

In some embodiments, the second data set includes the ultrasound imaging data.

In some embodiments, the first data set is used by the machine learning or deep learning operation (e.g., perform at the acquisition device) to output a classification output value selected from the group consisting of a classification code or identifier associated with no mass detected; a classification code or identifier associated a pre-existing, known-benign mass; a classification code or identifier associated with a new mass; a classification code or identifier associated with a known, confirmed cancer.

In another aspect, a system is disclosed configured to perform any of the above-discussed methods, wherein the system is a distributed local controller configured to keep a local cache of data relevant only to a given collection center for a pre-defined time duration or scans, wherein the acquisition devices can acquire and operate without network connectivity or connection to a central database for the pre-defined time duration or scans.

In some embodiments, the system includes cellular and/or Wifi-network interface.

In some embodiments, the system further includes at least one of a router, VPN, firewall, gateway, and bridge.

In some embodiments, the system further includes a Web server and/or a DICOM server.

In another aspect, an enterprise software system (e.g., SureView) is configured to perform or used with any of the above-discussed methods, the enterprise software system comprising a cloud-based clinical database (e.g., in a data warehouse) and processing.

In some embodiments, the enterprise software system includes an examiner quality monitoring module.

In some embodiments, the enterprise software system further includes one or more modules selected from the group consisting of: a teleradiology interface module; a remote examiner training module; an exportable electronic record module; a device integrity monitoring module; a management & billing information module; and a device/fleet firmware and software update module.

In another aspect, a non-transitory computer-readable medium is disclosed having instructions stored thereon, wherein execution of the instructions by a processor causes the processor to perform any of the above-discussed methods.

### BRIEF DESCRIPTION OF THE DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures.

FIG. 1A is a diagram of an example pressure elastography system that comprises a local cloud infrastructure and regional/global cloud infrastructure for pressure elastography measurements and management in accordance with an illustrative embodiment.

FIG. 1B is a diagram of another example pressure elastography system that comprises a local cloud infrastructure for pressure elastography measurements and management in accordance with an illustrative embodiment.

FIG. 1C is a diagram of another example pressure elastography system that comprises a regional/global cloud infrastructure for pressure elastography measurements and management in accordance with an illustrative embodiment.

FIG. 2A shows an example set of cloud services that may be executed in the cloud infrastructure of FIG. 1A, 1B, or 1C in accordance with an illustrative embodiment.

FIG. 2B shows an example global de-identified clinical database that may be executed in the cloud infrastructure of FIG. 1A, 1B, or 1C in accordance with an illustrative embodiment.

FIG. 3 shows an example operation of a local cloud infrastructure and the regional/global cloud infrastructure of FIG. 1A, 1B, or 1C in accordance with an illustrative embodiment.

FIG. 4A shows an example data collection appliance and gateway device that can implement the local cloud infrastructure of FIG. 1A or 1B in accordance with an illustrative embodiment.

FIG. 4B shows an example software implementation of the local cloud infrastructure, e.g., of FIGS. 1A and 1B, in the data collection appliance and gateway device of FIG. 4A in accordance with an illustrative embodiment.

FIG. 4C shows an example software implementation of the regional/global cloud infrastructure, e.g., of FIGS. 1A and 1C, in a data center or server in accordance with an illustrative embodiment.

FIGS. 5A and 5B are diagrams each showing an example workflow of a pressure elastography system comprising cloud infrastructure in accordance with an illustrative embodiment.

FIG. 5C is a diagram showing an example workflow between a pressure elastography system and an ultrasound system comprising cloud infrastructure in accordance with an illustrative embodiment.

FIGS. 6A and 6B each shows an example user interface for the local controller of a pressure elastography system and/or ultrasound system in accordance with an illustrative embodiment.

FIGS. 6C and 6D show another example display and visualization that may be generated by the local controller of the pressure elastography system and cloud infrastructure in accordance with an illustrative embodiment.

### DETAILED SPECIFICATION

Each and every feature described herein, and each and every combination of two or more of such features is included within the scope of the present invention provided that the features included in such a combination are not mutually inconsistent.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” (or superscript) corresponds to the nth reference in the list. All references cited and discussed in this specification are incorporated herein by reference in their entireties and to the same extent as if each reference was individually incorporated by reference.

While the application focuses on pressure elastography devices and ultrasound devices, it is contemplated that various components of the disclosure herein (e.g., local appliance and local cloud infrastructure) can be implemented for other imaging or medical modalities to provide electronic medical record capabilities, appointment management capabilities, as well as teleradiology interface capabilities for such modalities (e.g., MRI scanners, CT scanners, X-ray scanners, etc.). In some implementation, the local appliance and local cloud infrastructure described herein can be implemented for a surgical room or patient hospital room. In some implementation, the local appliance and local cloud infrastructure described herein can be implemented for diagnostic instruments (e.g., analyzers).

### Example System #1

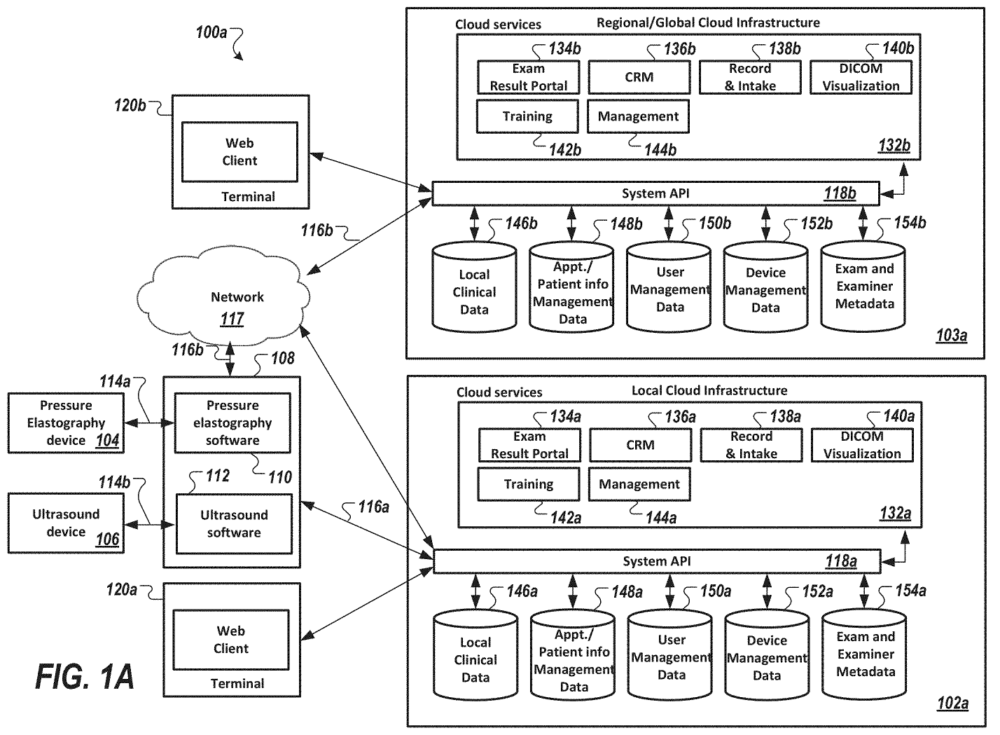

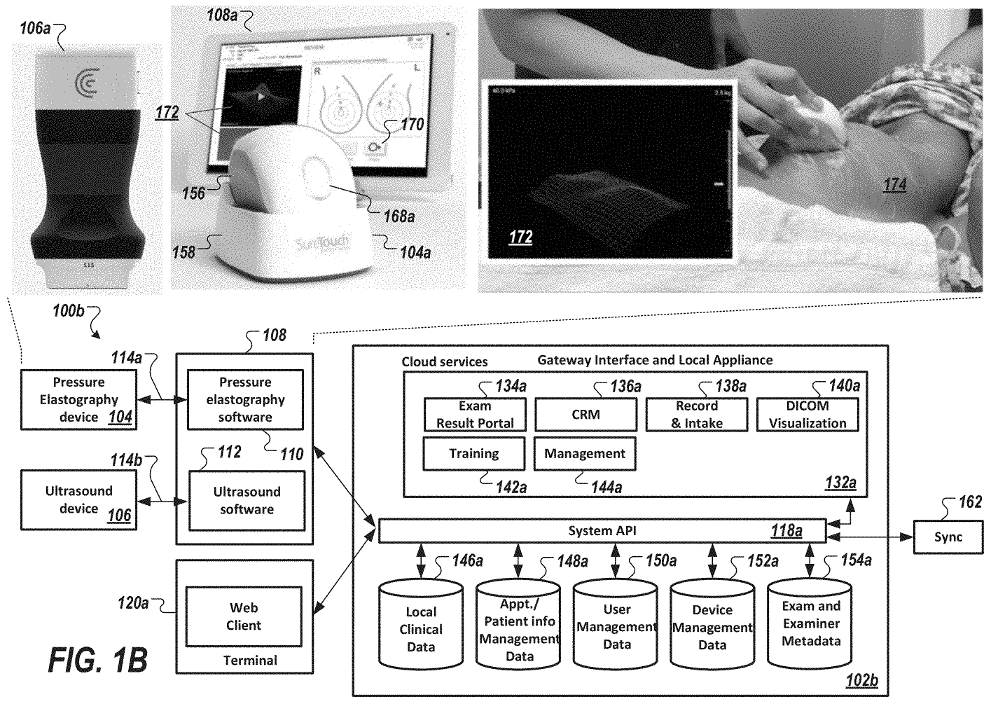

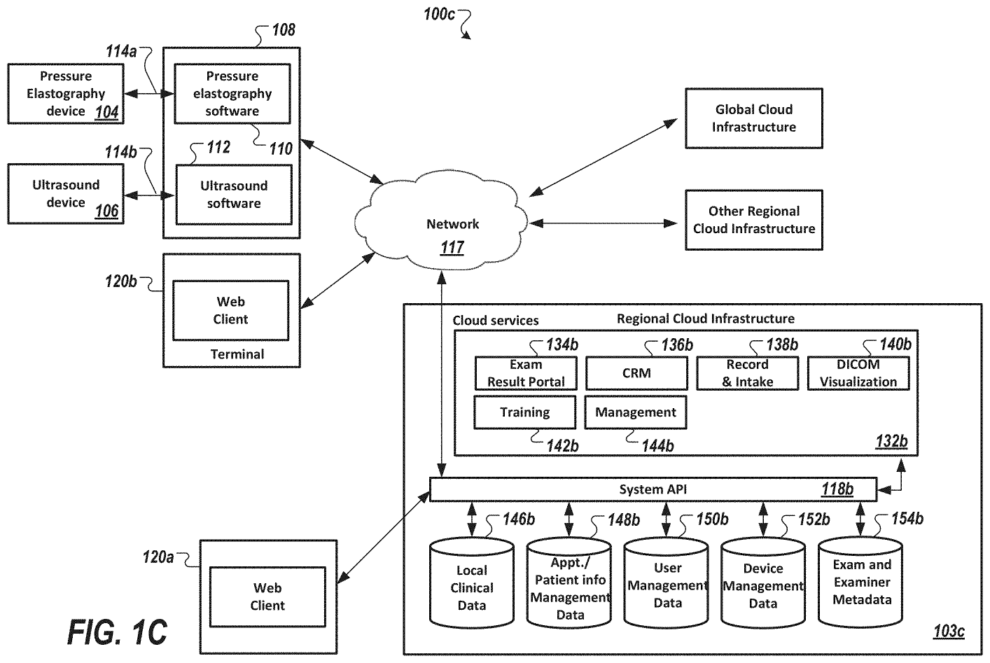

FIG. 1A is a diagram of an example pressure elastography system 100 (shown as 100a) that comprises a local cloud infrastructure 102 (shown as 102a) and regional/global cloud infrastructure 103 (shown as 103a) for pressure elastography measurements and management in accordance with an illustrative embodiment. FIG. 1B is a diagram of another example pressure elastography system 100 (shown as 100b) that comprises a local cloud infrastructure 102 (shown as 102b) for pressure elastography measurements and management in accordance with an illustrative embodiment. FIG. 1C is a diagram of another example pressure elastography system 100 (shown as 100c) that comprises a regional/global cloud infrastructure 103 (shown as 103c) for pressure elastography measurements and management in accordance with an illustrative embodiment.

Referring to FIG. 1A, the example pressure elastography system 100a includes a local cloud infrastructure 102a and a regional/global cloud infrastructure 103a that can, individually or in combination, provide management, collection, quality assessment, and optimized diagnostic workflow operations for pressure elastography measurements in accordance with an illustrative embodiment. System 100a includes a set of clinical measurement devices, including a pressure elastography device 104 (shown as 104a) and an ultrasound device 106 (shown as 106a). Examples of measurement devices 104, 106 are shown in FIG. 1B. The pressure elastography device 104 and ultrasound device 106 are operatively connected to a local controller 108 that locally stores the acquired clinical data, and that provides a viewer and interface to locally present data and control the measurement devices 104, 106. Following a scan, the local controller 108 provides the clinical data over a local network 116a to a local appliance that executes the local cloud infrastructure 102a to provide electronic medical record capabilities, appointment management capabilities, as well as teleradiology interface capabilities. The local controller 108 can also interface 116b over an external network 117 to a global or remote cloud infrastructure 103a that can provide the same electronic medical record capabilities, appointment management capabilities, and teleradiology interface capabilities. The local cloud infrastructure 102a and global cloud infrastructure 103a can synchronize and update to one another any data received there.

The local cloud infrastructure 102a through the local appliance can provide appointment management capabilities prior to the scan. Clinicians or office staff can schedule appointments for a patient through the local cloud infrastructure 102a or the global cloud infrastructure 103a and assign the patient a patient identification number. The patient can separately provide patient information and patient medical history through a patient portal comprising an intake interface provided through cloud services (e.g., as web services) that are hosted by the local cloud infrastructure 102a or the global cloud infrastructure 103a. The intake interface may be executed as a web interface, e.g., on a web client, executing at a client device 120 (shown as “Terminal” device 120a). The terminal device 120a can be implemented on any computing device, such as a laptop, smart phone, tablet, or personal computer. Then, during the start of the session, the clinician can access the appointment through the local controller 108 using the appointment information (e.g., time or appointment slot) or patient identification number. The appointment is linked to the patient information and patient medical history, as well as prior patient data and medical history, which can be associated with the appointment information or the patient identification number.

The term “clinician” (also referred to herein as an examiner or user) refers to a medical practitioner in a clinic, hospital, or doctor's office that is registered (and/or certified) to acquire the pressure elastography and/or ultrasound measurements. The database (e.g., 150) may maintain authentication information (e.g., username and password, roles, permissions) for a given registered user.

The local controller 108 includes a processor and memory having instructions to execute a pressure elastography acquisition module 110 and an ultrasound acquisition module 112. Modules 110, 112 provide the control interface for the pressure elastography device 104 and ultrasound device 106. Modules 110, 112 can also provide feedback information to the clinician. Pressure elastography measurement protocol/guidelines may require the consistent application of pressure applied to the patient during a scan. Module 110 can provide visualization of a real-time pressure monitor feedback display to the clinician. Module 110 can also provide visualization of examination data and summary, the pressure elastography scan, and interface to perform focused recording, e.g., when a mass is detected. Module 112 can provide the control interface for ultrasonic imaging, e.g., change parameters such as gain and depth, freeze and unfreeze images, change imaging modes, and view ultrasound scan data.

In some embodiments, the local controller 108 is implemented as a tablet or other mobile or portable computing device, e.g., with a touchscreen interface. In other embodiments, the local controller 108 is implemented in a mobile computing device or laptop, e.g., that is coupled or integrated into a medical cart. The connection 114a, 114b between the devices (e.g., 104, 106) and the local controller 108 may be a wireless communication such as Bluetooth, WiFi, ZigBee, or other low-power communication protocol. In other embodiments, the connection 114a, 114b may employ a proprietary communication protocol, or it can be a physical connection established over a cable, e.g., using a serial communication protocol. The local controller 108 is configured with a network interface to connect over a network channel 116 (shown as 116a) to the cloud infrastructure 102a through a set of APIs 118 (shown as “System API” 118a) that provides a number of management and collection operations (e.g., for device authentication, acquisition workflow, notification) for the cloud infrastructure 102a, 103a.

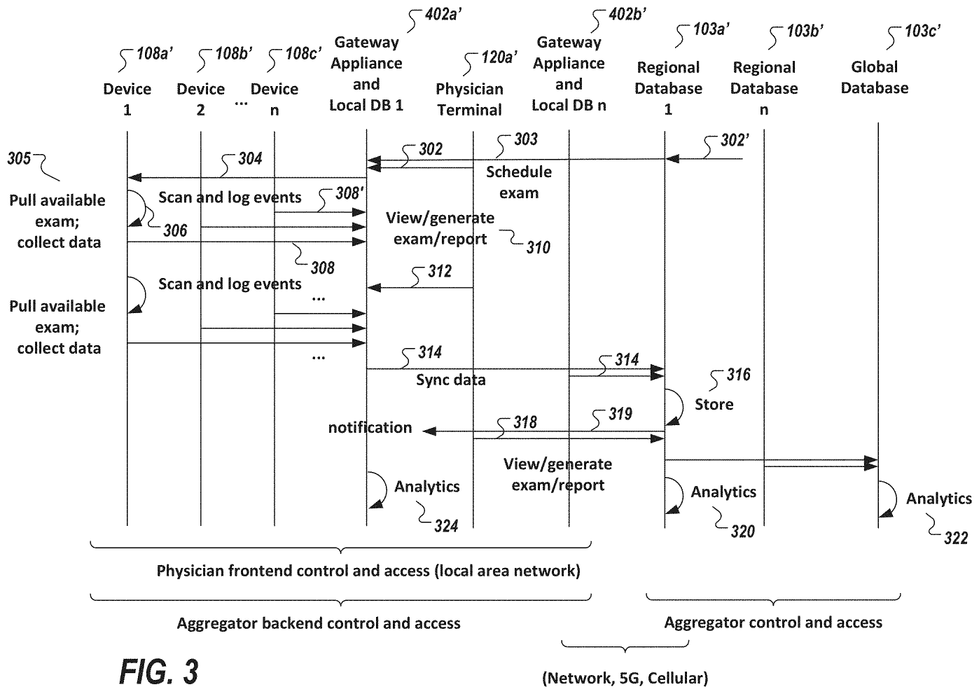

In some embodiments, the local controller 108 is configured to connect to the cloud infrastructure 102a executing on a data collection appliance and gateway device 402 (also referred to herein as a “SureSync Appliance”) (see FIG. 3). The local cloud infrastructure 102a can provide caching and site-specific management operation, e.g., to optimize the operation of multiple pressure elastography devices at a given geographic location or office. In some embodiments, the local cloud infrastructure 102a via the data collection appliance and gateway device 402 is configured to cache a given exam collection, e.g., when the network is unavailable. In some embodiments, the local cloud infrastructure 102a via the data collection appliance and gateway device 402 is configured to aggregate the exam collection and de-identify the exam data to provide a distributed acquisition with the global or remote cloud infrastructure.

Referring to FIG. 1A, the local cloud infrastructure 102a is configured to provide cloud services 132 (shown as 132a) (also referred to as “SureView™”), e.g., to provide a clinical exam interface 134 (shown as “Exam Result Portal” 134a) for transferring collected pressure elastography and/or ultrasound scans and reviewing pressure elastography scans (e.g., via a viewer), customer relation management (CRM) module 136 (shown as “CRM” module 136a), an electronic medical record, report generation, viewing, and patient intake module 138 (shown as “Record & Intake” module 138a), reviewing/viewing ultrasound scans 140 (shown as “DICOM Visualization” module 140a), a training module 142 (shown as 142a), and a device and user management module 144 (shown as 144a).

The regional/global cloud infrastructure 103a is configured with the same or similar cloud services 132 (shown as 132b), e.g., the clinical exam interface 134 (shown as 134b), the customer-relation management (CRM) module 136 (shown as 136b), the electronic medical record, report generation, viewing, and patient intake module 138 (shown as 138b), reviewing/viewing ultrasound scan module 140 (shown as 140b), the training module 142 (shown as 142b), and device and user management module 144 (shown as 144b).

In the example shown in FIG. 1A, the cloud infrastructure (e.g., 102a, 103a) are each configured to maintain a number of distributed databases, including exam data 146 (shown as “Local Clinical Data” 146a, 146b), patient data 148 (shown as “Appointment/patient information management data” 148a, 148b), enterprise user management data 150 (shown as 150a, 150b), device management data 152 (shown as 152a, 152b), and training data 154 (shown as “Exam and examiner Metadata” 154a, 154b).

Example Cloud Services

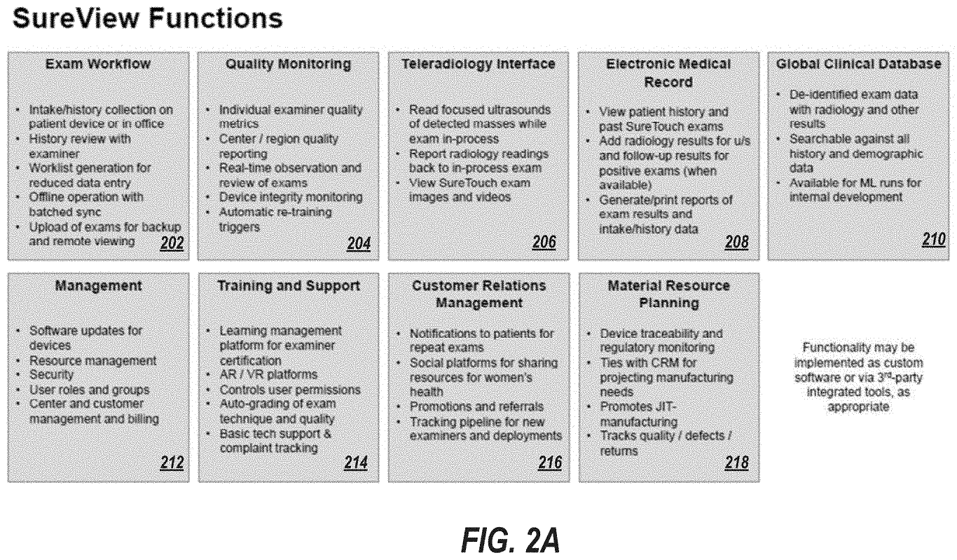

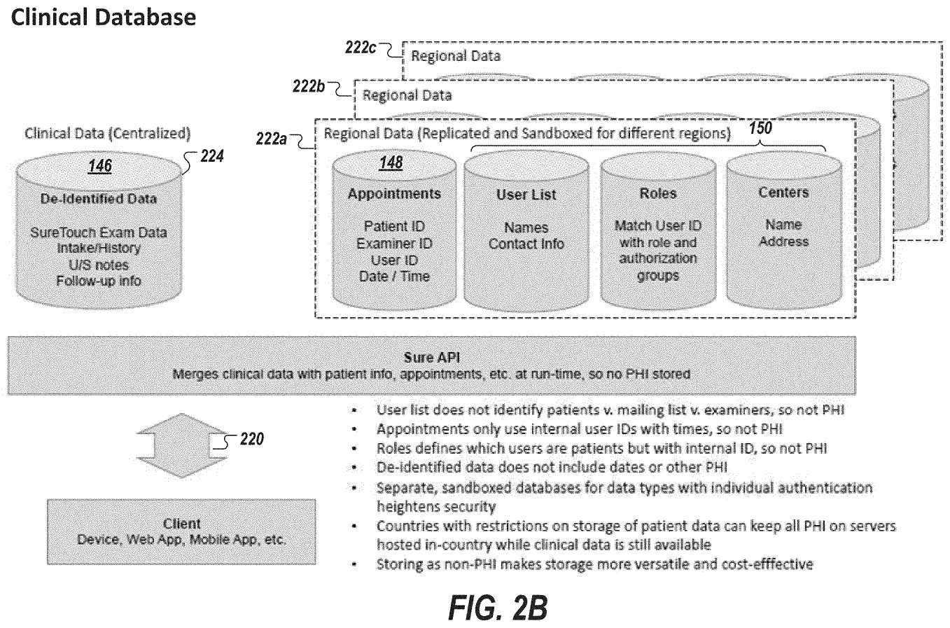

FIG. 2A shows an example set of cloud services (e.g., 132a, 132b) provided by the cloud infrastructure (e.g., 102a, 103a) in accordance with an illustrative embodiment. In FIG. 2A, the cloud infrastructure (e.g., 102a, 103b) may provide exam workflow operations 202 (e.g., via modules 138a, 138b), quality monitoring operations 204 (e.g., via modules 142a, 142b), teleradiology interface operations 206 (e.g., via modules 134a, 134b), electronic medical record operations 208 (e.g., via modules 134a, 134b), global clinical database operations 210 (e.g., via database 146a-154a and 146b-154b), management operations 212 (e.g., via modules 144a, 144b), training and support operations 214 (e.g., via modules 142a, 142b), customer relation management operations 216 (e.g., via modules 136a, 136b), and material resource planning operations 218 (e.g., via modules 144a, 144b). In some embodiments, these operations are implemented any number of modules, such as a teleradiology interface module, a remote examiner training module, an exportable electronic record module, a device integrity monitoring module, a management & billing information module, and a device/fleet firmware and software update module that are a part of the cloud services.

FIG. 2A is only an example embodiment, and other exemplary embodiments may be implemented. Certain cloud services (e.g., 132a, 132b) described in FIG. 2A may be optional in some implementation or may be implemented in external systems that interface to the device (e.g., 108) or to the cloud infrastructure (e.g., 102, 103). To this end, the cloud services (e.g., 132a, 132b) may be implemented in whole, or in some embodiments in part, in the cloud infrastructure (e.g., 102a, 103a) or may be distributed across the pressure elastography devices 104, local controller 108, and/or data collection appliance and gateway device 402, as well as with external devices.

Exam workflow operations 202. In some embodiments, the cloud infrastructure (e.g., 102a, 103a) may provide exam workflow operation 202, e.g., via modules 138a, 138b, to collect intake/history information of a patient. Modules 138a, 138b may provide web services that provide a portal for the patient to provide their information and medical history for a given appointment. Modules 138a, 138b can also provide web services that a portal that allows the clinician or examiner to review the collected intake/history information. Module 138a, 138b may also generate a worklist for a given user/examiner, e.g., based on appointments maintained by the system. This feature may be used to improve the workflow of the acquisition in reducing the data entry for a given appointment. Modules 138a, 138b may facilitate offline operation in being able to operate without access to a network and by collecting and batching exams after they are acquired and synchronizing them with the cloud infrastructure (e.g., 102a, 103a) once network connectivity is available.

Quality monitoring operations 204. In some embodiments, the local controller (e.g., 108) and cloud infrastructure (e.g., 102a, 103a) can perform quality monitoring and logging associated with the examiner and the exam. The quality assessment may be made from a combination of logging of measured metrics and statistics on the local controller (e.g., 108) and the reporting and analysis on the cloud infrastructure (e.g., 102a, 103a) for examiner monitoring, workflow monitoring, and/or device monitoring. The metrics can be first collected at the local controller (e.g., 108). Cloud infrastructure (e.g., 102a, 103a) may perform quality monitoring operations 204, e.g., via module 142a, 142b, to aggregate the metric for each given site (e.g., local cloud), each region (regional cloud), and globally (global cloud). Examples of quality metrics may include but are not limited to statistics associated with each exam, statistics associated with an examiner, statistics associated with a center or office, statistics associated with a region, statistics associated with a group or batch of devices and manufacturers.

Local controller (e.g., 108) can provide the monitoring of examiner and exam quality metrics by (i) logging events associated with user inputs at and state of the local controller (e.g., 108) and (ii) collecting exam statistics, e.g., relating to measured forces applied during the exam, various time durations performed during the exam, as well as exam findings and actions. The local controller (e.g., 108) can collect examiner and exam quality metrics for pressure elastography devices, ultrasound devices, or other acquisition devices integrated into the local controller (e.g., 108).

Table 1 shows a list of collected metrics associated with examiner monitoring.

TABLE 1

| Quality | |
|---|---|
| Metrics | Example of Collected Metrics |
| Exam and | Log of contact time for each breast, total contact time, |
| Examiner | exam time, discovery time, focused time, valid force time, |
| statistics | or time outside a pre-defined force range during an exam |
| | (in time and percent) |
| | Log of a number of recordings deleted in an exam |
| | Log of a number of re-recording during an exam |
| | Log of average applied exam force and variation for an |
| | exam |
| | Log of average discovery, focused force, and variation for |
| | an exam |
| | Log of edits made to settings, patient information, |
| | annotations, observations in an exam |
| | Log of actions performed on the local controller, e.g., |
| | changes in the state of operation of the local controller, |
| | invocation of functions of the local controller during an |
| | exam [event logging] |
| | Log of applied force applied by the examiner during the |
| | exam |
| Device | Log of battery levels over the course of the day (e.g., to |
| Statistics | identify an issue with hardware or excessive wear) |
| | Log of daily calibration data (e.g., to inspect sensor data or |
| | to identify broken electrical connection or device issues |
| | such as possible delamination of sensor layers) |
| | Log of an associated fleet of devices (e.g., based on sensor |
| | serial number, to identify quality issues with particular lots |
| | or assembly house) |

The examiner monitoring may be tied in with training/learning portal (e.g., labor-management partnership (LMP) initiatives) to recommend continuing education or re-certification as needed.

Metrics associated with workflow monitoring may be used to identify when significant deviations occur from training (i.e., from prescribed protocol or guidelines for the examination) to indicate a need to change recommendations or guidelines. For example, if a majority of examiners are consistently over or under the recommended force, then recommendations and acquisition guidelines may be re-evaluated. Similarly, recommendations and acquisition guidelines if the examination is observed to be too long or short per patient if there are large differences in the ratio of masses identified on the left or the right breasts. In some embodiments, the collected logs may be used to evaluate these metrics (e.g., how much time per patient/per breast changes) with the experience level of the examiner for the purpose of guiding training.

In some embodiments, the quality monitoring operation 202 may employ advanced algorithms (e.g., via machine learning) to assess such metrics, e.g., to identify and trigger when training or non-compliance of acquisition protocols or guidelines should be reported to an examiner or to a given center or office.

In some embodiments, the quality monitoring operation 202 is configured to obtain a first data set associated with a pressure elastography measurement acquired during a breast mass detection procedure conducted on a subject; analyze the first data set to determine one or more metrics associated with quality of acquisition, wherein the one or more metrics includes at least one of metrics associated with examiner monitoring, metrics associated with workflow monitoring, and metrics associated with device monitoring; and cause the one or more metrics to be outputted at a display or a report (e.g., for quality assessment of an examiner, a given device, a given workflow).

Teleradiology interface operations 206. In another aspect, to provide a more comprehensive scan for abnormal mass, the pressure elastography device 104 may operate with an ultrasound scanner to contemporaneously acquire an ultrasound exam to augment the pressure elastography measurement. The cloud infrastructure (e.g., 102a, 103a), in some embodiments, includes, in modules 134a, 134b, a data collection interface (e.g., for pressure elastography or ultrasonic scans) that can receive scan data collected in-process during an exam and push the read scan data to a teleradiology service. Modules 134a, 134b can also provide web services to present the exam results, including scan images, scan data set, and videos of the scan exam data. Results and reports from the reading can be pushed back, in some embodiments, to the local controller (e.g., 108) or cloud infrastructure (e.g., 102a, 103a) while the exam is in-process. In other embodiments, the report can be pushed to the cloud infrastructure (e.g., 102a, 103a) following the exam, which can trigger a notification for review by the clinician, examiner, or other office staff.

Electronic medical record operations 208. In another aspect, the cloud infrastructure 102a is configured, e.g., via modules 134a, 134b, to provide electronic medical record operations 208. Modules 134a, 134b can maintain and provide patient history and past exam records for a given patient in response to a record query. The past radiology results can be used for follow-up, e.g., where the exam determined a positive indication of a mass. The operation may be configured to generate and print reports of the exam results and intake/history data.

Global clinical database operations 210. In another aspect, the cloud infrastructure (e.g., 102a, 103a) is configured to maintain a global de-identified clinical database. The global de-identified clinical database may provide de-identified data to address regulatory conflict issues (e.g., local regulations) that may require protected health information (PHI) from being stored outside a given country or jurisdiction.

FIG. 2B shows an example global de-identified clinical database in accordance with an illustrative embodiment. In FIG. 2B, the system may separate the stored database data into multiple linked databases so that no single database contains records qualifying as PHI. In addition, in some embodiments, information from multiple databases may be combined in local server databases (e.g., at the data collection appliance and gateway) only when a query 220 is received from a client service of the cloud infrastructure (e.g., web app, device, or report generator) so that PHI is not stored on permanent media. The cloud infrastructure (e.g., 102a, 103a) may store de-identified clinical data 148 and interface with another set of databases 150 that maintains the patient identifier information (e.g., patient names, appointment times, etc. can be stored on local servers within a particular region if required by local regulations). The clinical data 148 can include only the scan data, patient intake information (without the patient name), exam notes, and follow-up notes. The clinical data 148 may be aggregated into a single centralized database (224), e.g., at the regional/cloud infrastructure (e.g., 103a), while the other patient information is maintained in specific regional cloud infrastructure (shown as 222a, 222b, 222c) to which the patient is located.

In the example shown in FIG. 1A, the cloud infrastructure 102 is configured to maintain a number of distributed databases, including exam data 146 (shown as “Local Clinical Data” 146a), patient data 148 (shown as “Appointment/patient information management data” 148a, enterprise user management data 150 (shown as 150a), device management data 152 (shown as 152a), and training data 154 (shown as “Exam and examiner Metadata” 154a). Exam data 146, in some embodiments, is stored in a first distributed database and include de-identified data that include ST clinical exam data. Patient data 148, in some embodiments, is stored in a second distributed database and include separately stored intake/history, U/S notes, and follow-up information, patient health information, including the patient's name, contact information, and appointment information. Enterprise user data 150, in some embodiments, is stored in a third distributed database and include user management information associated with the acquisition of the measurement, including username, password, role, and permissions of a registered clinician. Device management data 152, in some embodiments, is stored in a fourth distributed database and includes data associated with the management of the device. The data may include, but is not limited to, quality metrics associated with the device (e.g., up-time), manufacturing history, logistic history. Training data 154, in some embodiments, is stored in a fifth distributed database and includes data associated with the training of the user/examiner. The data may include collected examiner metrics and training modules. The examiner metrics may be used to assess the quality of the examiner to improve the accuracy of the measurement.

The separate databases (e.g., 146, 148, 150, 152, 154) can ensure that the user list does not identify patient, examiner, or patient addresses or associated information, e.g., billing. In addition, in some embodiments, appointments can be identified only by internal user identifiers and time. In some embodiments, roles parameters can include a label for examiner and patients in which the label is an internal identification number. In some embodiments, the databases can maintain a separate table for dates. In certain implementations, for regions with restrictions on the storage of patient data, the PHI data can be hosted on servers within that region.

Management operations (212). Referring to FIG. 2A, management operation 212 can be provided at the pressure elastography device 104, ultrasound device 106, local controller 108, data collection appliance and gateway 402, and cloud infrastructure (102a, 103a).

The cloud infrastructure (e.g., 102a, 103a), in some embodiments, is configured to provide remote IT services for software, firmware, and application updates for the pressure elastography device, local controller, and/or data collection appliance and gateway. In some embodiments, the cloud infrastructure (e.g., 102a) is configured, e.g., via modules 144a, 144b, to provide software updates for devices (e.g., 104, 106) that can be pushed to the device through the local controller (e.g., 108). In some embodiments, the software updates for the device are pushed to the local controller (e.g., 108) to be then pushed by a user to the device (e.g., 104, 106).

In some embodiments, Modules 144a, 144b can also provide resource management operation, security controls, user management, and permissions (e.g., to configure user roles and groups; configure center and customer information), and/or billing.

Training and support operations (214). In another aspect, the cloud infrastructure (e.g., 102a, 103a) is configured, e.g., via modules 142a, 142b, to provide training and support operations 214, e.g., to support operation and complaint tracking. In some embodiments, the cloud infrastructure (e.g., 102a, 103a) is configured to provide or host learning modules for user/examiner certification. In some embodiments, the learning modules may include AR/VR platforms (augmented reality/virtual reality) or an interface to such platforms.

In some embodiments, Modules 142a, 142b may include learning and/or certification submodules that are configured to record and grade a new examiner based on exam guidelines and collected quality metrics.

Customer relation management operations (216). In another aspect, the cloud infrastructure (e.g., 102a, 103b) is configured, e.g., via modules 136a, 136b, to provide customer-relation management operations 216. Modules 136a, 136b can be configured to send notifications to patients for repeat exams (e.g., based on results or based on pre-defined schedules). Modules 136a, 136b can maintain data for promotions and referrals. Modules 136a, 136b can track pipeline for new examiners and new device deployment. Modules 136a, 136b can also be web hosting or social platform hosting, e.g., for sharing resources on health and administrative functions.

Material resource planning operations (218). In another aspect, the cloud infrastructure (e.g., 102a, 103a) is configured, e.g., via Modules 144a, 144b, to provide material resource planning operations 218, e.g., to track quality, defects, and returns. Modules 144a, 144b can provide tracking of devices by their manufactured batch, manufacturer, and deployment dates. Modules 144a, 144b can be tied with the CRM operations (216) to provide a projection of manufacturing needs and inventory adjustments, e.g., to facilitate JIT-manufacturing. Modules 144a, 144b may provide tracking for devices (e.g., 104, 106) as well as accessories for such devices (e.g., gloves, lubricates, and other expendables).

### Example Systems #2 and #3

As discussed above, the exemplary system comprising the local appliance can be configured with the same, or substantially similar, implementation as the regional or global cloud infrastructure. While FIG. 1A shows the pressure elastography system being configured to operate with both a local cloud infrastructure (e.g., 102a) through a local appliance (e.g., 402) and a regional/global cloud infrastructure (e.g., 103a), the system can operate this infrastructure independently, e.g., when the network is unavailable, when the infrastructure is being deployed in locations without wide-area network connectivity (e.g., in remote locations). As discussed above, FIG. 1B is a diagram of another example pressure elastography system 100 (shown as 100b) that comprises a local cloud infrastructure 102 for pressure elastography measurements and management in accordance with an illustrative embodiment, and FIG. 1C is a diagram of another example pressure elastography system 100 (shown as 100c) that comprises a regional cloud infrastructure 103 (shown as 103c) for pressure elastography measurements and management in accordance with an illustrative embodiment. This doppelganger or twin capabilities provide for a seamless and unified operation that allows clinicians to interchangeably access clinical data, patient information, and other information between the local cloud infrastructure of the appliance and the regional or global cloud infrastructure, to provide cloud-based electronic medical record capabilities, appointment management capabilities, as well as teleradiology interface capabilities when network connectivity is unavailable.

As shown in FIG. 1B, the local controller 108 can independently operate, e.g., to provide functions described in relation to FIG. 2A, only with a local cloud infrastructure 102 (shown as 102b), e.g., executing on a local appliance (e.g., 402). In this configuration, the pressure elastography system 100b can be deployed at a remote location lacking network connectivity while having a fully contained infrastructure to provide cloud-based electronic medical record capabilities, appointment management capabilities, as well as teleradiology interface capabilities.

The system 100b can be deployed, for example, to a remote site or a set of remote sites for a prescribed duration, e.g., weeks or months. While deployed, system 100b provides pressure elastography and/or ultrasound scans, e.g., for breast cancer examination or screening, as well as local network connectivity and cloud services to manage the workflow of the examination. The system 100b is fully contained and can generate physical reports following scans for that site and maintain all the data and examination logs for subsequent transfer to a more permanent infrastructure and for analytics. To this end, after deployment, the system 100b can reconnect to the network (shown as “Sync” 162) and offload the data to regional/global cloud infrastructure (e.g., 103a) to extend, e.g., the electronic medical record capabilities, to a more permanent infrastructure and also for regional analytics, monitoring, and compliance tracking.

As shown in FIG. 1C, the local controller 108 can independently operate, e.g., to provide functions described in relation to FIG. 2A, only with a regional cloud infrastructure 103 (shown as 103c). Indeed, the local cloud infrastructure (e.g., 102a), while providing a set of benefits to workflow as described herein as well as redundancy, is optional to the operation of the regional cloud infrastructure (e.g., 103a).

Referring to FIG. 2A, examples of the pressure elastography device 104 (shown as 104a), ultrasound device 106 (shown as 106a), and local controller 108 (shown as 108a) are also shown, e.g., for remote deployment operation. Similar hardware can also be deployed for more permanent sites, e.g., doctor offices or clinics. In the example shown in FIG. 2A, the pressure elastography device 104a includes a handheld device 156 (also referred to as a mobile wand) and a dock 158 that can charge (e.g., wireless charge) the handheld device 156. The pressure elastography device 104a (also referred to as a “SureTouch™” or “Brexa™” device) can communicate to the local controller 108a implemented as a tablet to control the data acquisition, display, and analysis via the pressure elastography software (e.g., executing on Android OS) and be the primary interface to the pressure elastography device. The local controller 108a can be configured to communicate with the devices (e.g., 104a, 106a), e.g., over Bluetooth or WiFi communication. The pressure elastography device 104a can be an FDA-cleared unique, high-resolution pressure elastography device for bedside use in a clinical setting by trained personnel.

The pressure elastography device 104a, i.e., device 156, includes a capacitive sensor array, e.g., comprising a series of overlapping horizontal and vertical electrodes that form individual capacitors at each intersection. The capacitive sensing array is shielded for proper operation and to electrically isolate the patient from the device during the exam. The pressure elastography device does not administer, exchange, or supply energy to the patient, nor does it emit any ionizing radiation. The individual elements in the capacitive sensing array are capable of accurately detecting small differences in tissue pressure. Capacitive sensors have effectively no moving parts and suffer minimal fatigue during use, and so produce highly consistent, reproducible results while requiring very infrequent recalibration. The pressure elastography device function is based on abnormal masses in the breast being harder than the surrounding tissue so that when the capacitive sensor applies pressure to the breast, abnormal masses will produce higher response pressures, which can be used to generate an image of the mass and analyzed for estimates of the size and relative hardness.

The pressure elastography device 104a may be configured with a rechargeable lithium-polymer battery (not shown), a pushbutton 168 to start and stop recordings, and LED light output for power and status feedback. The local controller 108a can include one or more inputs 170 that provide selectable device or scan settings and/or selectable exam mode for the pressure elastography device 104a. The pressure elastography device 104a can employ high-resolution pressure elastography to detect and measure the size, shape, and relative hardness of masses found in the breast during an exam. The handheld sensor 156 can be used to examine the breast with the scan being transmitted to the local controller 108a comprising a tablet display to show the elastography images (e.g., 172). The local controller (e.g., 108a) can also display ultrasound scans and provide an interface to the ultrasound device 106a. In some configurations, the wand and tablet can be configured as rechargeable devices that can be used either while connected to wall power or can operate for an 8-hour shift from the rechargeable batteries in each component.

For each pressure elastography exam, a disposable sheath may be applied to the sensor (e.g., 156) and a lubricant applied to the breast to reduce friction during the exam. The examiner can then glide the sensor (e.g., 156) back and forth over the breast (see, e.g., patient 174) while applying light pressure and watching the display to monitor the applied force and identify any masses. If a mass is found, the examiner can direct the sensor (e.g., 156) to record data at that location for analysis (e.g., 5-10 seconds recording) using the touchscreen display to select a record mode. The pressure elastography acquisition module (e.g., 110) can analyze the data to display a visual of the mass and, in some embodiments, provide measurements associated with mass, e.g., an estimate of the size and relative hardness of the mass. At the end of the exam, the local controller (e.g., 108a) can signal the local cloud infrastructure (e.g., 102b) at the end of the exam. The local cloud infrastructure (e.g., 102b) can receive the scan data and summary and generate a report that summarizes exam results. The local cloud infrastructure (e.g., 102b), e.g., executing on the local appliance (e.g., 402), can print a hard copy of the exam report to provide to the patient. Patients with new masses can be further evaluated or referred to a second screening, e.g., ultrasound, diagnostic mammography, or digital breast tomosynthesis. In some embodiments, the second screening can be performed immediately, e.g., using the ultrasound device 106, following the pressure elastography scan, e.g., as described later herein.

Additional description and examples of the pressure elastography device 104a may be found in U.S. Pat. No. 6,500,119B1, U.S. Pat. No. 6,179,790B1, U.S. Pat. No. 6,468,231B2, U.S. Pat. No. 6,595,933B2, U.S. Pat. No. 7,419,376B2, U.S. Patent Publication no. 20040254503A1, U.S. Patent Publication no. 20040267121A1, U.S. Pat. Nos. 7,430,925, and 7,378,856B2, each of which is incorporated by reference herein in its entirety.

In addition to the wand and display, several accessories may be included with the system 100b, e.g., wand stand; sensor charger/AC adapter; display charger/AC adapter; system carrying case; calibration and training pad; and calibration scale. In addition to the included accessories, a lubricating lotion and disposable cover are included the system for use with the pressure elastography device. The lotion, which serves as a lubricant so that the sensor glides smoothly over the surface of the breast, may be any legally-marketed water-based, hypoallergenic, fragrance- and dye-free lotion or gel. The disposable cover may be any commercially-available, soft, biocompatible, legally-marketed disposable cover no more than 0.010 in. thick and at least 2.5×3.0 in. in size.

In the example of FIG. 2A, the ultrasound device 106a may be configured to acquire high-frequency linear ultrasound images (e.g., 5-15 MHz) of abnormal breast masses. The procedure may be performed in 15 minutes following or in conjunction with the pressure elastography measurement. An example of the ultrasound device 106a is L15 HD manufactured by Clarius Mobile Health Corp. (Burnaby, Calif.). The ultrasound device 106a can be configured to perform B-Mode, Color Doppler, and/or M-Mode scans.

During operation, the local controller 108 may be configured to generate an event log comprising a timestamped list of key events during an exam, e.g., screen change, start/stop recording, mass finding identified, patient data edited, among others. The event log can be provided to the local cloud infrastructure 102a, e.g., as 154a. Sensor log is a timestamped list of exam forces measured by the sensor throughout the exam, along with other diagnostic information. By comparing and combining these logs, the system can calculate and display many different types of quality metrics associated with the exam or examiner.

Example Synchronization Between Local Cloud Infrastructure and Regional/Global Cloud Infrastructure

FIG. 3 shows an example operation of the local cloud infrastructure (e.g., 102) and the regional/global cloud infrastructure (e.g., 103). As shown in the example of FIG. 3, the system can include one or more local controllers 108 (shown as “Device 1” 108a′, “Device 2” 108b′, and “Device n” 108c′), one or more cloud infrastructure 102 (shown executing on a “Gateway Appliance and Local DB 1” 402a′ and a “Gateway Applicant and Local DB 2” 402b′), and a set of regional and global infrastructure 103 (shown executing a “Regional Database 1” 103a′, “Regional Database n” 103b′, and “Global Database” 103c′).

In the example shown in FIG. 3, the local cloud infrastructure 102 executing on the “Gateway Appliance and Local DB 1” 402a′ can first be used to schedule 302 an examination. The client device 120 (shown as “Physician Terminal” 120a′) can access a cloud service application (e.g., 134a) hosted by the local cloud infrastructure 102 executing on the Gateway Appliance and Local DB “1” 402a′. As shown in FIG. 3, the appointment service application can be provided through the local cloud infrastructure 102 or the regional cloud infrastructure 103. When the appointment is made 302′ at the regional cloud infrastructure 103 executing the regional database “1” 103a′, the regional cloud infrastructure 103 then synchronizes 303 to the local cloud infrastructure 102, e.g., by pushing the appointment data to the local cloud infrastructure 102. The patient can provide patient intake information for the appointment, including their medical history, current medications, current medical or physiological status, and patient notes or comments prior to the examination, e.g., at home or at the clinic or doctor's office.

When the patient is being scanned, the appointment (e.g., for a scheduled examiner, site location, and/or time) is retrieved 304 from the local cloud infrastructure executing on the Gateway Appliance and Local DB “1” 402a′, and in some instances from the regional cloud infrastructure executing the Regional Database “1” 103a′. The clinician can log into the local cloud infrastructure executing on the Gateway Appliance and Local DB “1” 402a′ or other cloud infrastructure and find the appointment for the patient from a list of appointments. The clinician can then initiate 305 a scan by first acquiring a set of pressure elastography measurements.

During the exam 306, the clinician can prepare the sensor by applying a disposable sheath and lubricant. The clinician can then glide the sensor back and forth over the breast while applying light pressure (e.g., about 1 kg pressure) and watching the display to monitor the applied force and identify any masses. During the scan, the local controller 108a′ can provide feedback, e.g., of the applied force, to the clinician. The applied force data can be stored and provided to the cloud infrastructure (e.g., 102, 103) for compliance/documentation as well subsequent analysis (e.g., training). If a mass is found, the clinician can use the sensor to focus on that point and use the touchscreen display to initiate a recording at that location (e.g., 5-10 seconds recording). At the end of the exam, the clinician can select the scan complete button on the touchscreen display (e.g., of 108a′). The local controller 108a′ can log the time duration of the examination, the number of examinations taken for a given appointment, and other metadata or events associated with the examination as described herein. The local controller 108a′ can then signal 308 the local cloud infrastructure executing at the Gateway Appliance and Local DB “1” 402a′ and transmit the recorded exam data to the local cloud infrastructure. The local cloud infrastructure, e.g., through the Gateway Appliance and Local DB “1” 402a′, can direct the generation 310 of a report that summarizes exam results and direct, e.g., by printing, a hard copy of the exam report to give to the patient and/or to include in the physical patient medical records. Patients with new masses can be further evaluated or referred to a second screening, e.g., ultrasound, diagnostic mammography, or digital breast tomosynthesis. The local cloud infrastructure executing on the Gateway Appliance and Local DB “1” 402a′ is configured to receive 308 clinical data comprising the pressure elastography scan as well clinician data and event logs associated with the examination. That local cloud infrastructure can receive 308, 308′ the data from multiple local controllers 108a′, 108b′, 108c′ for a given doctor's office or clinic.

To view the examination results, a viewer application, e.g., executing at the “Physician Terminal” 120a′, that interfaces with module 134a can access 312 the local cloud infrastructure 102, e.g., executing at the Gateway Appliance and Local DB “1” 402a′. The data can also be curated or made accessible to the patient, e.g., provided the scan can be added to the patient medical history or record maintained by the doctor's office.

Subsequent to one or more scans being performed and provided to the Gateway Appliance and Local DB “1” 402a′ or other gateway appliances 402b′ (step 308), the Gateway Appliance and Local DB (e.g., 402a′, 402b′) can synchronize (314) to a respective regional/global cloud infrastructure executing the Regional Database “1” 103a′, e.g., by pushing the patient clinical data and the clinician's examination metadata to the Regional Database “1” 103a′. The regional/global cloud infrastructure executing the regional database (e.g., 103a′, 103b′) can store (316) the transmitted data, e.g., for subsequent viewing 318 or analytics 320.

For viewing operation, once the regional/global cloud infrastructure executing the regional database (e.g., 103a′, 103b′) receives and stores the transmitted data, the regional/global cloud infrastructure executing the Regional Database “1” 103a′ can send (319) a notification to the designated users. The notification can include a link or access information for the stored data. The user can then access 318 the stored data to view the clinical data, e.g., hosted by module 134a.

The regional database (e.g., 103a′ or 103b′) can provide the data or analyzed data to the global data 103c′ for analytics 322. As described herein, the clinical data are de-identified of patient data and include only a patient only. The clinical data are also maintained separately in a database separate from the database maintaining the patient information and medical history. The degree of analysis and data that can be performed at the regional cloud infrastructures (e.g., at operation 320) and at the global infrastructure (e.g., at operation 322) can be dictated by local and regional rules for a given country.

The Gateway Appliance and Local DB (e.g., 402a′, 402b′) can also perform local analytics (324), e.g., on the clinical data or on the exam/examiner, e.g., for quality monitoring or training. The analyzed data or the raw data of the Gateway Appliance and Local DB (e.g., 402a′, 402b′) can be provided to the regional databases (e.g., 103a′, 103b′) or to the global databases (e.g., 103c′) for storage or subsequent analytics.

Example Data Collection Appliance and Gateway Device

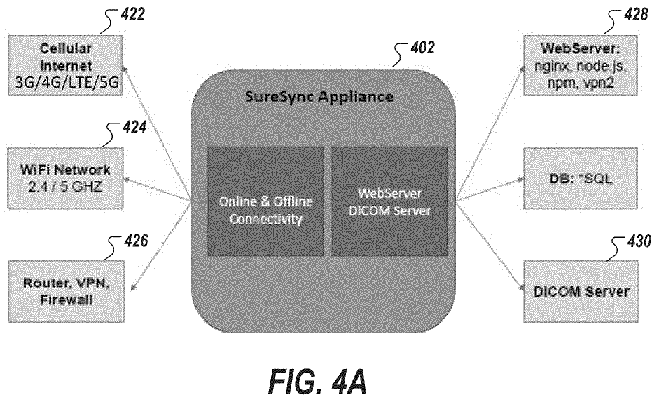

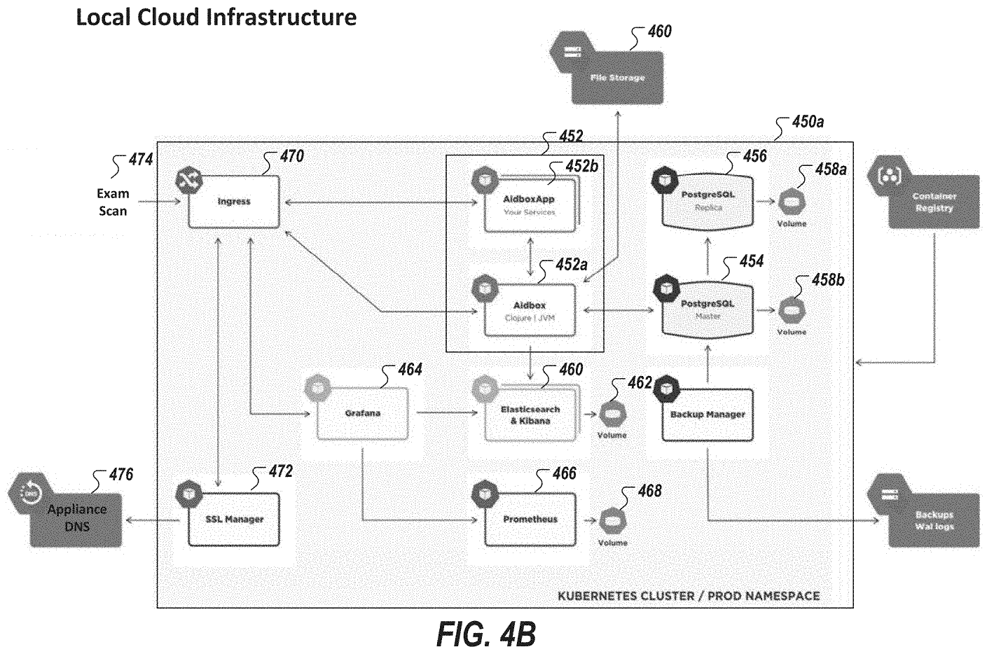

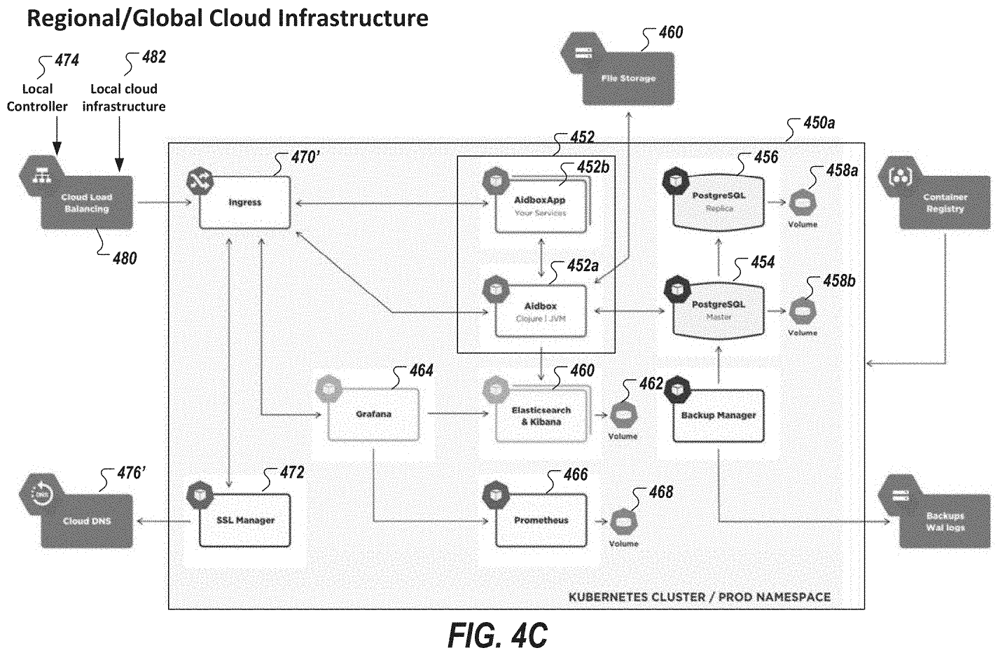

FIG. 4A shows an example data collection appliance and gateway device 402 (shown as “SureSync Appliance” 402) that can implement the local cloud infrastructure (e.g., 102) of FIG. 1A, in accordance with an illustrative embodiment. Other configurations for the data collection appliance and gateway device can be used, e.g., based on network/telecommunication availability, organization system, or user-cases.

As shown in the example of FIG. 4A, the data collection appliance and gateway device 402 is a distributed local cloud infrastructure that executes cloud services configured to keep a local cache of data relevant only to a given collection center for a pre-defined time duration or scans, where the acquisition devices (e.g., 104, 106) can acquire and operate without network connectivity or connection to a central database, e.g., of the regional/cloud infrastructure 103, for the pre-defined time duration or scans.

In some embodiments, the data collection appliance and gateway device includes a cellular interface 422 and a WiFi-network interface 424. The cellular interface can include 3G, 4G, LTE, 5G, or other communication technologies or protocols. In some embodiments, the data collection appliance and gateway device 402 is configured with anyone of a router, VPN, firewall, gateway, and bridge 426. In a preferred embodiment, the data collection appliance and gateway device 402 is configured with all of these features to operate as a standalone appliance that can provide local cloud services and infrastructure for electronic medical record capabilities, appointment management capabilities, as well as teleradiology interface capabilities. In some embodiments, the data collection appliance and gateway device 402 is configured with a Web server 128 and/or a DICOM server 130.

The data collection appliance and gateway device 402 can include a dual or multi-core processor (e.g., Intel or AMD (64 bit)) having two or more processing units configured to operate at, at least, 1.2 GHz, at least 8 GB of memory (Ram), and 1 TB of hard disk storage (HDD or SDD). The data collection appliance and gateway device 402 may have integrated 3G/4G/LTE/5G Cellular chipsets and/or antennas. The data collection appliance and gateway device 402 can also have WiFi router capabilities with 2.4 and 5 GHz Wireless broadband. In some embodiments, the data collection appliance and gateway device 402 can be implemented in a standard server configuration. In some embodiments, the data collection appliance and gateway device 402 is implemented in a portable or ruggedized workstation. In some embodiments, the data collection appliance and gateway device 402 is integrated into a medical cart that can house a set of local controllers (e.g., 108a) and measurement devices (e.g., 104a, 106a).

Example Software Implementation of Local Cloud Infrastructure

FIG. 4B shows an example software implementation of the local cloud infrastructure (e.g., 102) in the data collection appliance and gateway device (e.g., 402). FIG. 4C shows an example software implementation of the regional/global cloud infrastructure (e.g., 103) in a data center or server. In the example shown in FIGS. 4B and 4C, the local cloud infrastructure (e.g., 102) and regional/global cloud infrastructure (e.g., 103) can be implemented in a cluster 450 (shown as 450a and 450b, respectively) with similar or same components or services to provide for a seamless and unified operation that allows clinicians to interchangeably access clinical data, patient information, and other information between the local cloud infrastructure of the appliance and the regional or global cloud infrastructure. In addition, upgrades or modifications to the local appliance and gateway can be readily implemented in the doppelganger or twin regional or cloud infrastructure, reducing the cost of system maintenance and operation.

The local cloud infrastructure (e.g., 102) can execute in a cluster environment 450a. An example cluster environment (e.g., for 450a) is the Kubernetes (e.g., deployed by Cloud Native Computing Foundation). The cluster environment can be instantiated from a container-orchestration system having automated computer application deployment, scaling, and management capabilities. The cluster (e.g., 450a) can be deployed on a local appliance (e.g., 402) for the local cloud infrastructure (e.g., 102). The cluster (e.g., 450b) for the regional/global cloud infrastructure can be deployed on Amazon AWS, Google Cloud Platform, or Microsoft Azure.

The local cloud infrastructure (e.g., 102) can implement cloud service platform 452 that provides a secure environment for executing healthcare applications and/or health record exchange applications. An example of the cloud service platform 452 is Aidbox that is configured to execute a set of integrated patient-facing mobile applications. The cloud service platform 452 can include a base set of services 452a (shown as “Aidbox” 452a) and one or more healthcare application services 452b (shown as “AidboxApp” 452b). The healthcare application services 452b can include the clinical exam interface module 134, customer relation management (CRM) operations module 136, electronic medical record, report generation, viewing, and patient intake module 138, reviewing/viewing ultrasound scans module 140, training module 142, and device and user management module 144 as described in relation to FIG. 1A. In this context, the term “module” can refer to an application (e.g., healthcare software application).

The cloud service platform 452 can be configured using a Lisp-based programming language, e.g., Clojure. Clojure is a dynamic and functional dialect of the Lisp programming language on the Java platform that treats code as data and has a Lisp macro system. The applications (e.g., 134, 136, 138, 140, 142, 144) can be scripts or binary files that can be executed within the cloud service platform environment. The cloud service platform 452 can operate with one or more relational database management systems 454 (shown as “PostGreSQL Master” 454) and ba ack-up/duplicate relational database management system 456 (shown as “PostGreSQL Replica” 456). The relational database management systems 454, 456 can connect to one or more physical data storage shown having volumes 458a, 458b that can store the data tables for the exam data 146, patient data 148, enterprise user management data 150, device management data 152, and training data 154 as described in relation to FIG. 1A.

To improve the operation and management of the cloud service platform 452 and provide visualization of data by geographic location, the cloud infrastructure (e.g., 102, 103) can execute search and analytics engine, observability platform, and data visualization dashboard. In the example shown in FIGS. 4B and 4C, the cloud infrastructure (e.g., 102, 103) executing on cluster 450a, 450b can execute a distributed search and analytics engine 460 (shown as “Elasticsearch & Kibana” 460), e.g., for each instance of the base set of services 452a and the one or more healthcare application services 452b. The distributed search and analytics engine 460 can be used for log analytics, full-text search, security intelligence, business analytics, operational intelligence, among others. Elasticsearch is a distributed search and analytics engine that can be built on a search engine software library, e.g., Apache Lucene, and can be used for can provide such functionality and analysis for geospatial data in 2D or 3D geographical maps. Kibana is a data visualization dashboard software that can operate with the distributed search and analytics engine 460 to provide visualization of data in histograms, line graphs, pie charts, sunbursts, etc., of geospatial data in geographical maps, e.g., in ElasticMaps. The distributed search and analytics engine 460 can provide the log analytics, security intelligence, business analytics, and operational intelligence data to a data store shown as volume 462.

In the example shown in FIGS. 4B and 4C, the cloud infrastructure (e.g., 102, 103) can also execute an observability platform and agents 464 (shown as “Grafana” 464) that can provide logs, traces, dashboards, alerts, for each module or element of the cloud stack, including, e.g., hardware resource utilization (e.g., memory, network bandwidth), login history, search engine hits, cloud infrastructure performance (e.g., latency, throughputs, request received or serviced). These logs, traces, dashboards, alerts can be provided for each application, e.g., healthcare application services 452b, as well as other services executing in the cluster or platform.

In the example shown in FIGS. 4B and 4C, the cloud infrastructure (e.g., 102, 103) can also execute a monitoring and alert module 466 (shown as “Prometheus” 466) that can collect and store its metrics as time-series data, i.e., metrics information is stored with the timestamp at which it was recorded, alongside optional key-value pairs/labels. The monitoring and alert module 466 can collect the logs, traces, dashboards, alerts, e.g., as time-series data, and store them in a data storage volume 468.

In the example shown in FIGS. 4B and 4C, the cloud infrastructure (e.g., 102, 103) can execute an ingress module 470, e.g., executing system API 118a, to direct requests to add clinical data, examination event logs, and examiner data from a given scan. The ingress module 470 of the local cloud infrastructure (e.g., 102) can receive the clinical data file, examination event logs, and examiner data file 474 from, and acquired at, the local controller (e.g., 108). The ingress module 470 (shown as 470′) of the regional/global cloud infrastructure (e.g., 103) can receive the clinical data file, examination event logs, and examiner data file 474 from the local controller (e.g., 108) (e.g., shown as 474) or from the local cloud infrastructure (e.g., 102) (e.g., shown as 482), e.g., through a cloud load balancing module 480, e.g., that is a part of the Amazon AWS, Google Cloud Platform, or Microsoft Azure cloud infrastructure.

The cloud infrastructure (e.g., 102, 103) can also execute a secure egress module 472 (shown as “SSL Manager” 472), e.g., to direct requests (e.g., REST requests) to the appropriate cloud services, e.g., the clinical exam interface 134, customer relation management (CRM) operations 136, electronic medical record, report generation, viewing, and patient intake 138, reviewing/viewing ultrasound scan 140, training 142, and device and user management 144, e.g., as described in relation to FIG. 1A. The egress module 472 of the local cloud infrastructure (e.g., 102) can operate with the appliance DNS 476. The egress module 473 of the regional/global cloud infrastructure (e.g., 103) can operate with the appliance DNS 476′.

Example Workflow

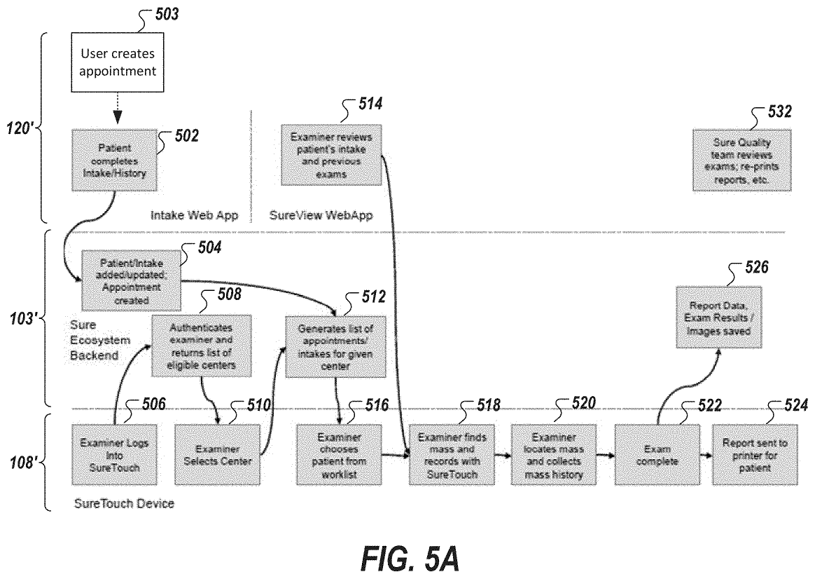

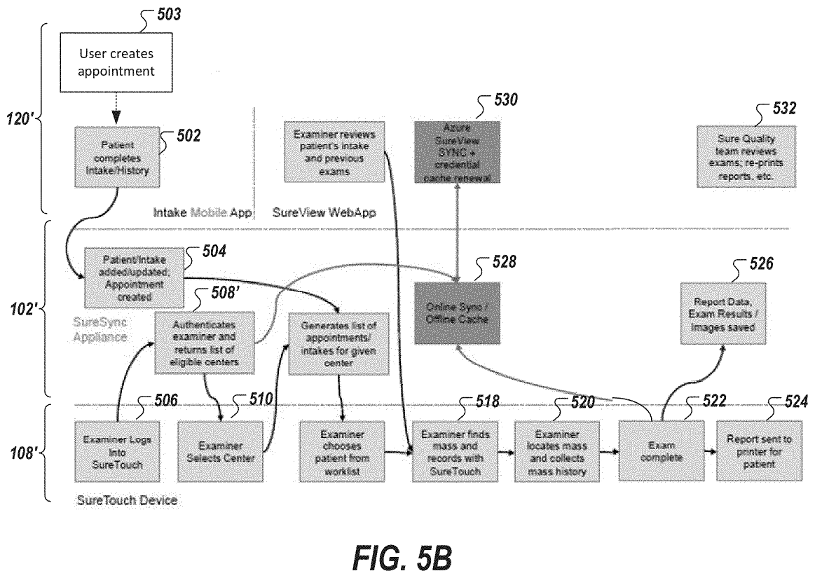

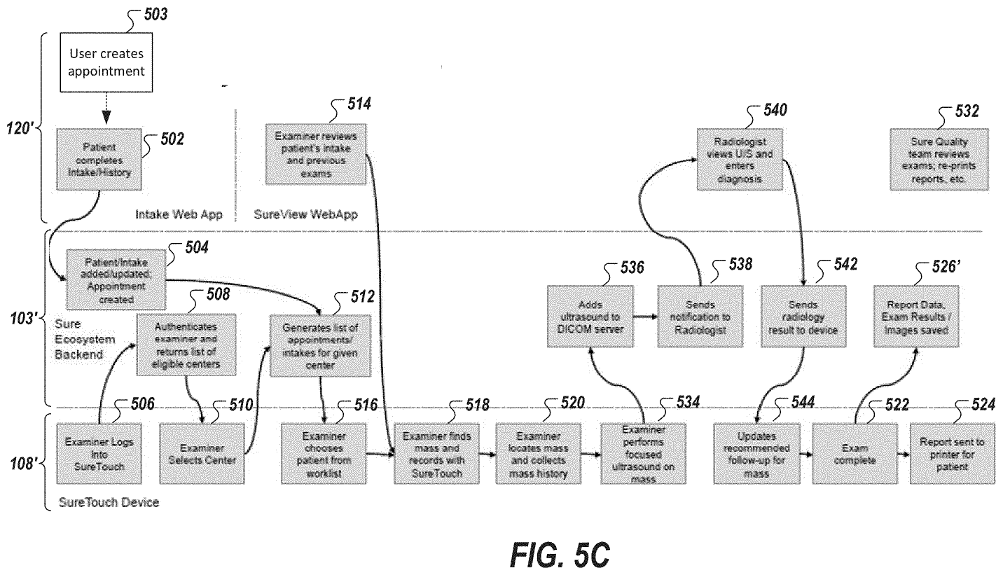

FIGS. 5A and 5B are diagrams each showing an example workflow between the pressure elastography system comprising a local controller 108 (shown as 108′), a terminal or remote computing device 120 (shown as 120′), and the regional/global cloud infrastructure 103 (shown as 103′) or the local cloud infrastructure 102 (shown as 102′), respectively, in accordance with an illustrative embodiment.

In the example shown in FIGS. 5A and 5B, the process includes a patient completing (502) an intake work/health form and providing requested medical history information, e.g., through an intake web interface (e.g., operating on a client device at a clinic, hospital, doctor office, etc.). The intake work/health form and relevant medical history information are used to generate or confirm 504 an appointment for a scan, which can be performed at the regional cloud infrastructure 103′ (FIG. 5A) or the local cloud infrastructure 102′) (FIG. 5B) executing at the data collection appliance and gateway device (402). In some embodiments, the appointment can be generated (503) by a user (e.g., clinician or office staff) at the clinics, hospitals, doctor's office to which the patient can provide (502) the intake information.

In the example shown in FIGS. 5A and 5B, a user/examiner can log (506) into the local controller 108′ of a pressure elastography system and authenticate to the regional cloud infrastructure 103′ (FIG. 5A) or to the local cloud infrastructure 102′ executing at the data collection appliance and gateway device (e.g., 402) to perform a scan of the patient. The cloud infrastructure (e.g., 102′ or 103′) is configured to provide (shown as 508 and 508′, respectively) authentication data to the pressure elastography system 108′ based on provided examiner credential (e.g., comprising an examiner identifier).

In the example shown in FIGS. 5A and 5B, the user/examiner may be prompted 510 to select a center/location for the scan and/or an associated appointment. The selection can be used to generate (512) a list of appointments to pre-populate (514) data fields of the exam or correlate the data collected from the intake operation (502) or the prior exam, e.g., the patient's information, based on a subsequent selection 516 of the appointment by the examiner. The scan (e.g., pressure elastography measurement) is then conducted (518) to screen or evaluate for a mass. If a mass is detected during the scan, the examiner can direct (520) using the touchscreen display the device (e.g., 104) to acquire a focus scan at the location of interest and record the measurement. A recording can be for 5-10 seconds in some implementation. The local controller (e.g., 108) can maintain the scan or recordings as time-series data or convert the data to a video or image file (e.g., healthcare video or image file format). Once the examination is complete, the examiner can indicate (522) the completion of the scan on the touchscreen display. At the conclusion of the exam, the cloud infrastructure 103′ or local controller 108′ may initiate (524) the generation of a report that includes the pressure elastography results. The results are transferred (526), e.g., through a network (e.g., 117), from the local controller 108′ to the cloud infrastructure 103′ (FIG. 5A) and stored there or through a local area network to the local cloud infrastructure 102′ (FIG. 5B) for storage and/or subsequent analysis there.

In FIG. 5A, following a scan (e.g., 520), the results can be pushed (526) to the regional/global cloud infrastructure 103′ for analysis, management, and etc. In FIG. 5B, following a scan, the results are pushed (526) to the local cloud infrastructure 102′ executing on the data collection appliance and gateway device (e.g., 402), which can cache (528) the results in batches to be transferred to the regional/global cloud infrastructure 103′, e.g., for analysis, management, and etc. The cloud services (e.g., executing on 102′ or 103′) can also perform synchronization (530) of the new results. In some embodiments, the collection appliance and gateway device (e.g., 402) may operate with a remote/external server (not shown) that can provide some of the analysis and/or management for the cloud infrastructure (e.g., 102′ or 103′).

Following the transfer of the scan to the cloud infrastructure (or remote server) (e.g., 522), the cloud infrastructure (e.g., 102′ or 103′) can provide teleradiology operation to push the result to appropriate tele- or local-radiology services to evaluate the scan and to push, or make available, the evaluation on the healthcare portal. An example operation is shown in FIG. 5C, which is discussed in further detail below. In the example of FIGS. 5A and 5B, the cloud infrastructure (e.g., 102′ or 103′) can also report 532 or perform analysis (e.g., machine learning-based analysis) of event logs and various metadata, including examiner's metadata described herein, generated during the examination.

Multi-Modality Acquisition

The multi-modality acquisition of the pressure elastography device, e.g., in conjunction with an ultrasound device, can be used to improve the accuracy of the diagnosis or to meet certain examination guidelines. FIG. 5C is a diagram showing an example workflow between the pressure elastography system and ultrasound system in accordance with an illustrative embodiment. In the example shown in FIG. 5C, following a focus scan and recording at a location of interest having a potential detected mass (e.g., 534), the examiner can conduct a second evaluation of the patient using the ultrasound device (e.g., 106). The local controller 108′ can include in its interface an input to initiate ultrasound acquisition. The examiner can then conduct (534) the ultrasound examination to review the mass detected by the pressure elastography system. Once the ultrasound examination (e.g., 534) is complete, the examiner can indicate the completion of the scan on the touchscreen display. The results (e.g., pressure elastography results and/or ultrasound results, e.g., depending on the system configuration) can be pushed (536) to the regional/global cloud infrastructure 103′ (FIG. 5C) for analysis, management, and etc., or to the local cloud infrastructure 102′ (not shown). The regional/global cloud infrastructure 103′ (FIG. 5C), or the local cloud infrastructure 102′ (not shown), e.g., executing modules 134a, 134b, can send 542 a notification to a teleradiologist, e.g., that can view (542) the results, as hosted by cloud services (e.g., 134, 140), through a client device (e.g., 120). The results from the teleradiologist can be received (542) at the regional/global cloud infrastructure 103′ (FIG. 5C), or the local cloud infrastructure 102′ (not shown), and recommendations can be presented (544) on the local controller 108′. In some embodiments, the results may be provided, i.e., presented to the patient and/or reviewed by the clinician, subsequent to the scan, and after the examination has been completed (not shown).

Local Controller Interface

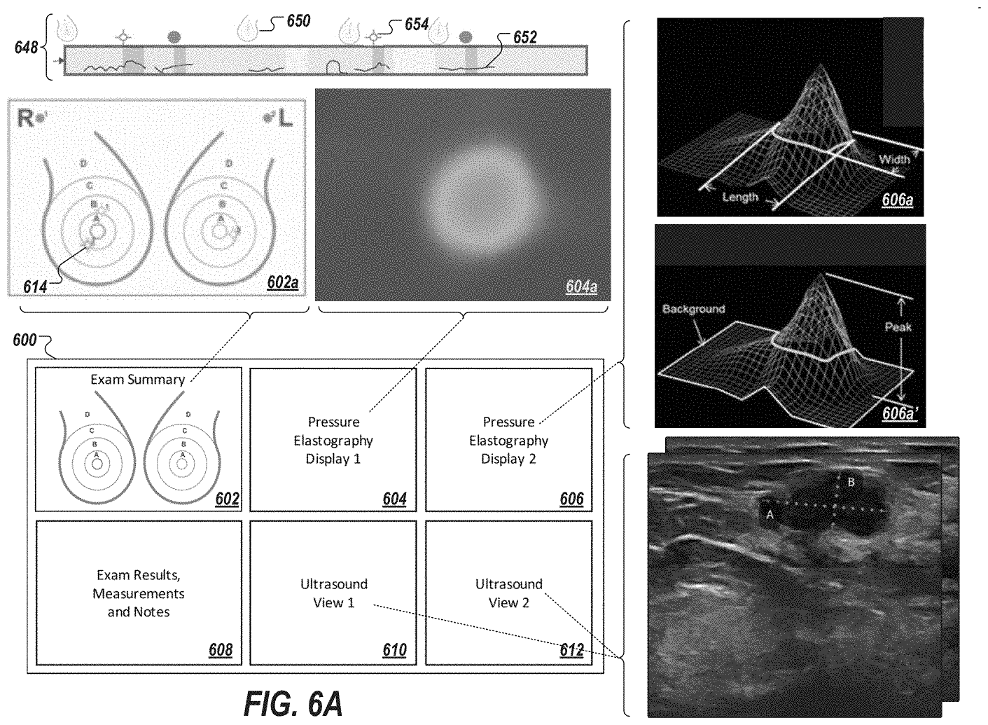

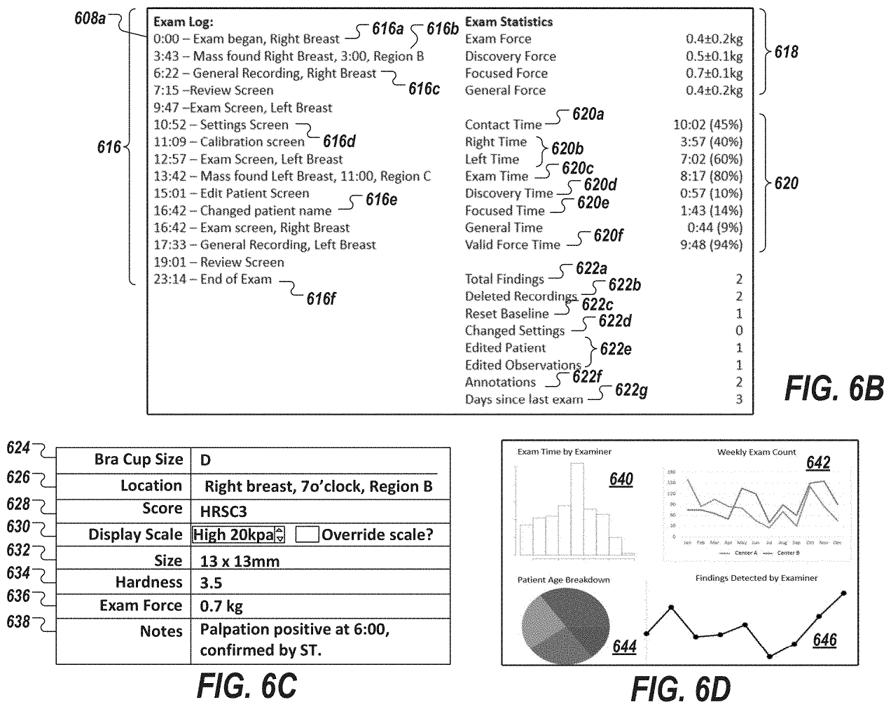

FIGS. 6A and 6B each shows an example user interface for the local controller (e.g., 108) in accordance with an illustrative embodiment. In FIG. 6A, the user interface 600, e.g., for the local controller (e.g., 108), may include one or more panels (shown as 602, 604, 606, 608, 610, and 612) to display and record the pressure elastography scan, including a first panel 602 to record a location of a potential mass. The panels can be consolidated or stratified to present the information described herein.

In the example of FIG. 6A, panel 602 shows a map of the left breast and the right breast. An example input is shown in panel 602a to record a location 614 of a detected mass during an acquisition. The spatial location of a detected mass can be inputted by the examiner (e.g., manually on the interface 602a), or it can be ascertained based on the scan and then automatically populated by the local controller (e.g., 108) on its interface. The user interface 600 can include buttons and/or graphical elements (not shown) to add that location 614 or to initiate recording mode. Multiple locations for different masses can be added to panel 602.

In the example shown in FIG. 6A, the user interface 600 can also include one or more panels (e.g., 604 and 606) to show pressure elastography measurements. In this example, a two-dimensional view of the pressure elastography measurement is shown in panel 604a, and a three-dimensional view of the pressure elastography measurement is shown in panel 606a. The two-dimensional view (e.g., panel 604a) and three-dimensional view (e.g., 606a) may be updated in real-time during the acquisition. The two-dimensional view (e.g., in panel 604a) may be color-coded in which the intensity of the presented color corresponds to the measured stiffness. The three-dimensional view (e.g., in panel 606a) may show the spatial information (e.g., shape/geometry) of the identified mass, e.g., at location 614, in which the x and y axes (panel 606a) corresponds to the area of the scan, and the height/peak corresponds to or indicates the maximum stiffness of that mass (panel 606a′). The user interface 600 also includes numerical measures of the scan and identified mass. It may also display the exam log as generated during the scan.

The user interface 600 may provide exam or examiner force feedback data to the examiner. In FIG. 6A, the user interface 600 may display a graphical summary 648 of the events logged (656) during the exam in conjunction with a visualization of the applied force. Different background shades can be used to indicate software mode. Icons (650) can be displayed to indicate specific events (e.g., finding identified or breast change). Lines (652) can be presented to indicate exam force when contact with the patient is detected.

FIG. 6B shows an example display of panel 608 (shown as 608a). The panel 608a includes the exam log 616, which can include events relating to the start of the exam (616a), the timestamp and location of a detected mass (616b), the start of a recording (616c), the current displayed views of the interface (616d), edits to the appointment or patient information (616e), and the end of the exam (6160. In essence, the local controller (e.g., 108) can log any input made to the interface 600, the sensor device (e.g., the buttons on the devices 104, 106) as detected by the controller, as well as any functions or monitoring operations, and their outputs, being executed on the controller (e.g., 108). The user interface 600 may also display exam statistics 618 (e.g., mean and standard deviation), the exam metadata 620 (e.g., total contact time (620a), contact time per breasts (620b), exam time (620c), time to a discovery of a mass (620d), focused recording time (620e), as well as valid force-time (6200). Indeed, the local controller (e.g., 108) can provide such exam statistics as part of exam quality as well as training and feedback for the examiner. The user interface 600 may also present exam metadata 622 for the examiner, e.g., the number of findings by the examiner (622a), the number of deleted recordings (622b), the number or frequency of resets of the baseline (622c), the frequency of adjustment of settings (622d), the frequency of edits to the patient, appointment, or observation information (622e), the frequency of additions of annotations (6220, frequency of use/exam conducted (622g).

FIG. 6C shows another example display of panel 608 (shown as 608b). The panel 608b can include clinical data regarding the scan, including, e.g., the bra cup size of the patient (624), the location of a detected mass (626), a mass score (628), the current display scale of the user interface (630), the size of the detected mass (632), the hardness of the detected mass (e.g., average or maximum hardness) (634), the applied force during the recording of the mass (636), and notes (638).

FIG. 6D shows an example visualization that may be generated by the cloud services of the local cloud infrastructure (e.g., 102) or regional/global cloud infrastructure (e.g., 103) through the aggregation of such exam or examiner metadata for a given examiner, office, or region. The cloud services can present the information for a specific period of time. The aggregated visualization can include exam time by examiners (640), weekly exam count for a given site (or monthly, quarterly) (642), the patient information (644) (e.g., age demographics, hometown, time period), and findings of masses (646) (e.g., by site location/office, examiner, time period).

In some embodiments, the local controller (e.g., 108) can be configured to provide a workflow that facilitates switching between pressure elastography measurement module (e.g., SureTouch app) and ultrasound app and allowing the ultrasound app to talk directly, e.g., to a DICOM server. In other embodiments, the local controller (e.g., 108) is configured to use an ultrasound API to collect and display data within the pressure elastography measurement module (e.g., SureTouch UI and app). The pressure elastography measurement module (e.g., SureView teleradiology viewer) may show ultrasound images alongside SureTouch images when available via existing, FDA-cleared DICOM viewer.

In some embodiments, when a mass is found and an ultrasound device (e.g., 106) is connected to the local controller (e.g., 108), the controller may generate a message to send to the cloud infrastructure for an on-call radiologist for review. The radiologist may review the ultrasound scan within a SureView interface (desktop or mobile) and indicates whether further work-up is needed. When consult results are posted, the cloud infrastructure may push a notification to the pressure elastography device or local controller to add radiology results to SureTouch data while the exam is still in-progress.

Discussion

Pressure Elastography (e.g., as provided by the SureTouch device), for example, has been reported to be able to find masses in 8-10% of women. Focused ultrasound may be used in conjunction with pressure elastography on the same masses to rule out 50% of those masses for a net referral rate of ˜4%. Ultrasound technicians/user/examiners may be readily rained to perform pressure elastography exams. In some embodiments, the ultrasound may be performed using a wireless ultrasound probe connected to the same local controller (e.g., a tablet running the SureTouch device software).

Breast cancer is the most common women's cancer and the leading cause of cancer death among women. Breast cancer is generally diagnosed through either screening or a symptom (e.g., pain or a palpable mass) that prompts a diagnostic exam. Screening of healthy women is associated with the detection of tumors that are smaller, have lower odds of metastasis, are more amenable to breast-conserving and limited axillary surgery, and are less likely to require chemotherapy[4], all scenarios with reduced treatment-related morbidity and improved survival. Detecting breast cancer at its early stages can also dramatically reduce the cost of care (COC) as the total COC for early-stage breast cancer (Stages 1& 2) is 50% less than the total COC for later stage breast cancers (Stage 3 & 4). However, the easiest means of reducing the impact of this deadly disease—effective early detection—has achieved only partial adoption in the United States and is largely absent when considered on a global scale [5]. Currently, in the United States, “early detection” and “screening” typically mean screening mammography (and rarely, whole breast ultrasound for the subset of women who have dense breast tissue, though whole breast ultrasound has never been widely deployed as a frontline screening modality). Breast examination by a health professional—while simple, inexpensive, widely available, and consistently adopted by women—has unfortunately been shown to perform poorly as a screening procedure.

Breast Mass Detection. 80% to 95% of invasive breast cancers present as a solid or cystic tumor within the breast [6,7]. A new, palpable breast mass—most commonly discovered during breast self-examination or clinical breast examination—is a common presenting sign of breast cancer and is the most common presentation of breast cancer in women not undergoing regular screening mammography due to age or personal choice. [8] Breast cancer may also present as a palpable mass in between mammographic screens, known as “interval cancer.” In general, cancers detected by palpation or because of symptoms like changes in the breast shape or skin are detected at later stages and therefore have a poorer prognosis than cancers detected by screening of asymptomatic patients [9].

Clinical Breast Exam. In the United States, the original first step into the early detection of breast cancer was a clinical breast exam (CBE), typically performed in a primary care setting by a physician or nurse. Because most breast masses do not exhibit distinctive physical findings that allow a determination of benign or malignant, imaging evaluation is necessary in almost all cases to characterize the palpable lesion. Commonly recommended imaging options in the context of a palpable mass include diagnostic mammography and breast ultrasound and are dependent on patient age, degree of radiologic suspicion, and radiologist preference. When a suspicious finding is identified during imaging, the biopsy is indicated [9]. The American College of Obstetricians and Gynecologists (ACOG) has recommended CBE in women aged 20-39 years old every 1-3 years and annually in women aged 40 years and older. ACOG, USPSTF, and ACS also promote “breast self-awareness.” [10,11] ACOG includes instruction for and encourages self-breast examinations for high-risk women, but they in the minority among medical professional organizations in doing so.

CBE has proved to be a relatively insensitive examination with large variation in effectiveness between clinicians. A retrospective review by van Dam et al. found that CBE has a positive predictive value of 73% and a negative predictive value of 87% [12]. The technique of CBE, even when performed in largescale studies, has generally not been standardized, which may partly be due to the highly subjective interpretation of CBE findings among clinicians. While a well-conducted CBE can detect up to 50% of cancers not detected by mammography alone, the overall performance in typical practice is much less uniform [13]. This has led multiple professional U.S. medical professional organizations to not recognize CBE as an efficacious means of early detection, effectively making screening mammography the universal entry point into early detection in the United States.

Screening Mammography. The only modality proven to reduce breast cancer-specific mortality is screening mammography [14]. Screening mammography leads to a 19% overall reduction in breast cancer mortality [15], with less benefit for women in their 40s (15%) and more benefit for women in their 60s (32%). As a result, screening mammography schedules have been developed and endorsed by Physician Societies, as reported in Table 2 below.

Diagnostic Mammography. Women presenting with a breast mass undergo diagnostic (as opposed to screening) mammography. Diagnostic mammography can be performed in women at any age; however, in women younger than 40 years old, the denser glandular breast tissue lowers the sensitivity, and therefore ultrasound directed at the area of concern is the preferred study.

Breast Ultrasonography. Breast ultrasonography complements diagnostic mammography in the evaluation of a palpable breast mass. It delineates the shape, borders, and acoustic properties of the mass. The primary use of ultrasonography is to distinguish cystic lesions from solid masses. Ultrasonography has other uses: assessment of the underlying cause of an abnormal finding on CBE, evaluation of a palpable breast mass in a young woman with dense breast tissue or a woman with breast implants, differentiation of poorly delineated masses as cystic or solid, and assessment of peripheral masses located outside the field of view of a mammogram. Ultrasonography can guide interventional procedures because the needle can be visualized continuously within the mass, ensuring an adequate sample¹⁶. The negative predictive value of mammography with ultrasound (US) in the context of a palpable mass ranges from 97.4% to 100% [17].

Current Guidelines for Detection and Diagnosis. Nowadays, due to the inconsistent performance of CBE as a means of early detection in a primary care setting and the lack of any alternative technology, ‘early detection’ has become synonymous with ‘screening mammography.’

Screening Mammography. Mammography was first introduced as an imaging modality as early as 1930 but was not immediately accepted as a useful modality or screening option until the 1960s based primarily on the work of Robert Egan and Raul Leborgne, with widespread adoption following in the early 1970s as it was introduced into the “war on cancer.” [18] The improvement in sensitivity of mammography over CBE has been widely credited with saving thousands of lives and reducing unnecessary morbidity, but the high rate of falsely positive exams has been a source of controversy since the early 1990s [19]. Screening mammography represents a substantial improvement in sensitivity over CBE, consistently demonstrating a sensitivity of 79%-80%, although there is a large variation in sensitivity, from 63% to 84%, across radiologists [20]. The specificity of screening mammograms has been among the primary drivers of changes in mammography screening guidelines. The false-positive rate of screening mammography is commonly cited at 12%, but difficult-to-interpret mammograms that are not formally “falsely positive” generate an additive number of referrals for additional procedures. Claims reviews from US-based insurers consistently demonstrate a total rate of referrals for additional imaging following screening mammograms of 14% to 21% (falsely positive results+difficult-to-interpret results) [21].

Guidelines for screening mammography represent efforts to balance the value of screening with 80% sensitivity against the risk, morbidity, and expense of unnecessary secondary imaging studies and biopsies and of repeated radiation exposure. As evidence has accumulated, guidelines have both evolved and become inconsistent among medical associations.

The American Cancer Society (ACS), U.S. Preventive Services Task Force (USPSTF), and American College of Radiology (ACR), and most academic imaging and oncology departments agree that annual mammographic screening starting at 40 years old saves the most lives [22, 23, 24]. However, debate over the interpretation of evidence, risk, cost and acceptability of additional studies, procedures and expense began in 1967 and continues today, with corresponding effects on screening mammography recommendations. The three main strategies for average-risk women are summarized in Table 2.

TABLE 2

| | American College of | | U.S. Prevention |
|---|---|---|---|
| Screening | Radiology, SBI, | American Cancer | Services Task Force, |
| Mammography | NCCN, NCBC, AsBrS | Society | AAFP, ACP |
| Initiation Age | Recommended at 40 y | Offer at 40 y-44 y; | Begin at 50 y; |
| | | recommended at 45 y | individual decision |
| | | | from 40 y-49 y |
| Internal | Annual | Annual 40 y-50 y; | Biennial |
| | | biennial or | |
| | | annual ≥55 y | |
| Cessation Age | Continue as long as | Continue as long as | Stop at 74 y; |
| | healthy and desire to | life expectancy | insufficient evidence |
| | be examined | is ≥10 y | to continue after 75 y |

While this debate remains unresolved, there are two practical impacts of these considerations. These guidelines are not formulated to save the most lives and reduce the morbidity of breast cancer; in the absence of a high frequency of unnecessary diagnostic procedures, and with a lower cost test, annual detection should be started at age 40 in every woman, and at age 35 in African American women whose average age at diagnosis is 3-4 years younger than the white and Hispanic populations [25]. Otherwise stated, these guidelines do not address the need for or value of early detection. Instead, they address the use of screening mammography.

Disagreement among professional societies causes confusion among women, which is a factor in non-compliance with screening recommendations.

Clinical Need. A combination of factors inherent in screening mammography has created a large and expanding “detection gap” defined as the population of women of at-risk ages for breast cancer who do not receive the benefit of effective annual screening mammography. An early detection examination that women will not comply with has zero predictive value and impact on the portion population of women who will not or do not have the examination.

The size of the detection gap is much larger than is commonly appreciated by laypersons or physicians. In the U.S., over 25 million eligible women, or 32% of women at at-risk ages for breast cancer, do not receive screening mammography [26], most commonly because they refuse the procedure for a number of reasons discussed below. Noncompliance is exaggerated in ethnic populations, and women in rural settings face an additional challenge of accessing a mammography provider.

Globally it is estimated that over 1 billion women will never have access to the protection of screening mammograms due primarily to the capital and operating costs of the equipment [27]. An additional portion of U.S. women is “sub-optimally screened” due to guidelines promoting the withdrawal of screening mammography from women 40-49 and/or biennial or lesser frequency of examination. The cost-benefit analysis of screening mammography that drives the withdrawal of mammography from this population also fails to suggest a replacement test even though breast cancer risk is best addressed with annual early detection beginning at age 40 (35 for African American women), and the only age group still seeing a growth in breast cancer frequency is women 40-44 [25,28].

Noncompliance. The largest portion of the detection gap results from women's refusal to have a mammogram for a variety of reported reasons, including the pain of breast compression, fear of radiation exposure, discomfort with intimidating medical facilities, and anxiety over delayed results of the procedure (typically 7-10 days) [29]. Conservatively, 31%, or 20.3 million, US women at the highest-risk ages will not consent to a mammogram or regular mammograms, and this number is understated in several ways. First, HEDIS scores are a common reference for screening compliance but do not include the 40 to 49-year-old population. Second, the studies of screening mammography participation often employ inconsistent and misleading inclusion criteria, such as “any mammogram in the previous 37 months,” all of which serve to inflate the participation rate. As an anecdotal data point, Sure spoke with a major US health insurance provider, who reviewed their claims and found that only 40% of enrolled women were receiving their recommended screening mammograms. While lack of education and awareness is a common compliance challenge in non-U.S. healthcare systems, decades of promotion, education & activism addressing the importance of breast cancer early detection has significantly reduced the lack of awareness as a factor in noncompliance.

A version of the procedure and experience of mammography is a universal limitation of the test. Mammography in Canada is freely available, even in Northwest Ontario, where transportation to local locations is an included service, yet only 34% of women avail themselves of organized screening mammography programs [30, 31]. Saudi Arabia has more mammography equipment per capita than any of the other wealthy countries in the region, and mammograms have always been free, yet only 8% of Saudi women are willing to have mammograms [32].

Gaps Left by Clinical Guidelines & Recommendations. Among clinicians, there is consensus that detecting breast cancer early can reduce morbidity and mortality and, further, that age 40 is the best age for white and Hispanic women to begin early detection [33]. The median age at diagnosis for African American women is 3-4 years younger than for white and Hispanic women, setting their age for the beginning of early detection at age 35 or 36 [25,34]. An annual frequency of early detection based upon the average growth rate of breast cancer is also considered to be the optimal frequency to best prevent “interval cancers” that occur between screening mammography examinations [35]. All these conclusions are based upon the age of onset and an average pace of growth of breast cancer. Guidelines for screening mammography vary from these facts because of limitations of the procedure of mammography, not because of changes in the pathogenesis of breast cancer affecting age at diagnosis or screening intervals. These guidelines work to compel women 40-49 into avoiding screening mammography until age 50 and then to have biennial examinations. There are 20 million women in the U.S. aged 40-49 that are losing access to screening mammography as their primary care physicians adopt these guidelines, and 58.9 million women 50+ that these guidelines are pushing to biennial mammograms [36].

Per the University of California San Francisco radiology department website [37], there is a benefit of screening, including a 39.6 percent mortality reduction from annual screening of women 40-84 years old vs. 23.2 percent mortality reduction if following USPSTF task force recommended biennial screening between ages 50-74. And, if all U.S. women currently aged 30-39 undergo annual mammography screening between ages 40-84, an estimated 100,000 more lives will be saved than if following the current USPSTF guidelines of the biennial (every two years) screening between ages 50-74.

Limited Access. 15.7 million American women aged 40 and above live-in rural communities [39]. Rural populations are burdened with higher rates of cancer incidence and cancer mortality and tend to have limited access to preventive screening services, especially services requiring proportionally high capital investment and operating expenses, including mammography [39, 40, 41, 42, 43, 44]. In rural areas, only 42% of African-American females receive regular screening mammograms, and 54% of white females receive regular screening mammograms, resulting in between 7 and 8 million women in rural areas receiving no screening mammograms and in the detection gap [45]. Research also indicates that women living in rural areas are less likely to receive mammograms at medically recommended time intervals [46, 47, 48, 49]. Challenges to access include not only physical distance but also the cost of travel, childcare, and absence from work.

Radiation Risks. Regarding radiation-induced breast cancers, their incidence, and mortality rates, Migloretti et al., in their 2016 Annals of Internal Medicine article, conclude that, relative to the number of deaths averted with (mammography) screening, radiation-induced breast cancer incidence is not trivial [53]. Their analysis projects 125 to 246 cases of radiation-induced breast cancer with 16 to 32 deaths per hundred thousand women, with higher risk levels for women receiving annual screening (versus biennial) and with large breasts or implants that require extra views. They conclude that the benefit-to-harm ratio in terms of breast cancer deaths averted per radiation-induced breast cancer could be improved by initiating screening [mammography] at age 50 years instead of 40 years. In the absence of radiation risk and to achieve the goal of early detection, examinations should begin at age 40. Their conclusion is appropriately driving changes in mammography recommendations, in doing so expanding the population of women in the detection gap.

Global Lack of Early Detection. Globally, very few women will ever receive the benefit of screening mammography. The average penetration of screening mammography into the global at-risk population is approximately 5% or a detection gap of 1.3 billion women. Assessed on a global scale, lack of access to mammography and lack of education and are the primary bases for this screening gap; there are also large regions where cultural factors like aversion to exposed physical examination and male physicians involved in the screening process conspire to reinforce and perpetuate the avoidance of traditional screening.

A Large Detection Gap. Among the population of women of common at-risk ages for the most common women's cancer, breast cancer, most women beginning at age 40, and African-American women beginning at age 35, there are a large gap of women who do not receive the protection of effective annual early detection: (i) Women who refuse screening mammography, (ii) Women in low income and rural settings who lack access to mammography, (iii) Women 40 to 49 of normal risk who are advised against annual mammograms, and (iv) Women 50+ who are advised to have mammograms every two years, and African American women 35-39.

This gap translates to between 40 and 70 million women in the U.S. receiving no or suboptimal early detection and over 1 billion women worldwide who are not receiving any sort of early detection for breast cancer. This gap is only amplified by the current COVID-19 pandemic, which has further eliminated options for early detection for millions of women.

It is important to note that the term ‘gap’ in ‘detection gap’ refers to the difference between the medically-recommended regimen for early detection of breast cancer and the complex and inconsistent guidelines for screening mammography based on a difficult cost-benefit analysis involving a variety of factors outside the simple efficacy of the exam. In the ideal case, all women would have access to an annual exam for early detection, which could bring them into existing recommended diagnostic pathways.

Background on Elastography. Elastography is the creation of an image or ‘map’ (technically an ‘elastogram’) of different tissues based upon their varying elastic properties. Elastography produces both quantitative data in the form of measurements of tissue elasticity, either measured relative to adjacent tissues or estimates of absolute elasticity, as well as the size and positional data, and qualitative data in the form of images created from elasticity measurements.

Tissues being evaluated for their elastic nature are most accurately described as “more elastic” or “more inelastic,” but in practice, the terms ‘stiffness’ and, more commonly, ‘hardness,’ are used to describe comparative tissue elasticity. While these terms are not technically interchangeable, they are commonly used as synonyms with ‘stiffer’ and ‘harder’ meaning ‘less elastic,’ and ‘softer’ meaning ‘more elastic.’ These terms are used interchangeably herein.

Simply stated, the elastic nature of tissue can be evaluated by two methods: by measuring the response of the tissue to a compressive force or by measuring the response of a tissue to a specific type of ultrasound called ‘shear wave.’ The use of a compressive force to evaluate elasticity is commonly called ‘pressure elastography’ and is the type of elastography employed by a pressure elastography device (e.g., SureTouch device manufactured by Sure, Inc. of Los Angeles, Calif.).

These most common modalities used to measure elasticity operate in different fashions, but each measures the same elastic properties of tissue. Pressure imaging is also called ‘mechanical,’ palpation,′ or ‘tactile’ imaging and uses passive pressure sensors that emit no energy but instead measure the “pushback” from underlying tissues as they are compressed. Shear-wave ultrasound produces a specific type of sound wave that is projected into the tissue to derive information based upon elasticity. Both forms of elastography can produce a visual representation of the shape of tissues and a quantitative estimation of their hardness, either as an estimation of actual hardness or as the hardness of tissue relative to adjacent or nearby tissues.

It has been well established that abnormal tissues, including malignant tissues, are less elastic, or ‘harder,’ than most normal tissues, primarily based upon differences in cellular architecture and vascularity, and that malignant tumors are particularly hard compared to all other normal and abnormal tissues [61, 62, 63, 64, 65, 66, 67]. This is the primary basis for using elastography to detect and identify abnormal tissues including masses, both benign and malignant.

While pressure elastography is a new bedside technology, elastography has been performed and studied for more than 30 years [64, 65]. As an imaging modality, elastography has been focused by practicality upon solid organ tissues that frequently require an evaluation of internal tissues and structure, and then with organs that are physically accessible by ultrasound or pressure sensor probes. Liver, breast, and prostate examinations for internal structural abnormalities indicating disease or tumor have been most commonly studied, and shear wave elastography of the liver has proven to be a useful clinical tool, now performed by technology dedicated to that examination and commercially produced for routine clinical use, with elastography of the liver currently bearing a Category 1 CPT code and routinely reimbursed by insurance providers.

Elastography of the breast to detect breast cancer has been a source of great clinical interest given (1) that breast cancer is the most common women's cancer and (2) that existing methods for the early detection of breast cancer suffer from suboptimal performance characteristics, serious side effects, and adoption limited to only a portion of the women at risk.

Evaluation of breast tissue by elastography is appealing because there is no obstruction to the examination of the entire breast, including the tail of Spence, by a handheld sensor, given the structure of the breast as a mass of tissue attached to the relatively smooth underlying chest wall. The tail of the breast is challenging to evaluate with traditional mammography, yet up to 20% of breast cancers originate in the tail of the breast.

Many studies of breast imaging using the shear-wave ultrasound have been and continue to be published. Most often, these studies examine the ability of shear wave elastography to differentiate cancer from benign masses [68, 69, 70].

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

Computer system capable of executing the software components described herein for the local controller and data collection appliance and gateway can include specialized or standard hardware. The computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that is not directly bound to the number of computers in the computing device. In an embodiment, the functionality disclosed above may be provided by executing the application and/or applications in a cloud computing environment. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing device typically includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing device may have additional features/functionality. For example, computing device may include additional storage such as removable storage and non-removable storage including, but not limited to, magnetic or optical disks or tapes. Computing device may also contain network connection(s) that allow the device to communicate with other devices such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device may also have input device(s) such as keyboards, keypads, switches, dials, mice, track balls, touch screens, voice recognizers, card readers, paper tape readers, or other well-known input devices. Output device(s) such as printers, video monitors, liquid crystal displays (LCDs), touch screen displays, displays, speakers, etc. may also be included. The additional devices may be connected to the bus in order to facilitate communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include, but is not limited to, volatile media, non-volatile media, removable media and non-removable media implemented in any method or technology for storage of information such as computer readable instructions, data structures, program modules or other data. System memory, removable storage, and non-removable storage are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art. It is also contemplated that the computer architecture may not include all of the described components and may include other components that are not described.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Further examples of processing that may be used with the exemplified method and system are described in U.S. Pat. No. 6,500,119B1, U.S. Pat. No. 6,179,790B1, U.S. Pat. No. 6,468,231B2, U.S. Pat. No. 6,595,933B2, U.S. Pat. No. 7,419,376B2, U.S. Patent Publication no. 20040254503A1, U.S. Patent Publication no. 20040267121A1, U.S. Pat. Nos. 7,430,925, and 7,378,856B2, each of which is incorporated by reference herein in its entirety.

Moreover, the various components may be in communication via wireless and/or hardwire or other desirable and available communication means, systems and hardware. Moreover, various components and modules may be substituted with other modules or components that provide similar functions.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
[1] Harvey S, Gallagher A M, Nolan M, Hughes C M, “Listening to Women: Expectations and Experiences in Breast Imaging,” Journal of Women's Health, 2015 Sep. 1, 24(9); 777-783.
[2] Wilson R, “COVID-19 Sidelines Breast Cancer Screening,” Oncology Times, 2020 Apr. 20, 42(8): 14.
[3] “Special Report: COVID-19's Impact on Breast Cancer Care,” https://www.breastcancer.org/treatment/covid-19-and-breast-cancer-care, 2020 Jul. 29.
[4] Fuller M S, Lee C I, Elmore J G. Breast cancer screening: an evidence-based update. Med Clin North Am. 2015; 99:451-468.
[5] Laxminarayan, R, Chow, J, Shahid-Salles, S A. “Intervention Cost-Effectiveness: Overview of Main Messages.” In: Disease Control Priorities in Developing Countries, 2nd Edition by D. T. Jamison, et al., 2016. Oxford University Press. 36-86.
[6] Dillon D, Guidi A J, Schnitt S J. Chapter 25: Pathology of Invasive Breast Cancer, in Harris J R, Lippman M E, Morrow M, Osborne C K. Diseases of the Breast, 5th edition, Lippincott Williams & Wilkins, 2014.
[7] Joe B N. Clinical features, diagnosis, and staging of newly diagnosed breast cancer. In: UpToDate. Burstein H, Vora S R (eds.). Waltham, Mass.: UpToDate, 2019.
[8] Mathis K L et al., “Palpable Presentation of Breast Cancer Persists in the Era of Screening Mammography,” J Am Coll Surg, 2010 March, 210(3): 314-318.
[9] Expert Panel on Breast Imaging: Linda Moy, et al., ACR Appropriateness Criteria® Palpable Breast Masses, J Am Coll Radiol. 2017 May; 14(5S):5203-5224.
[10] Final Recommendation Statement—Breast Cancer: Screening. U.S. Preventive Services Task Force. November 2016. https://www.uspreventitiveservicetaskforce.org/Page/Document/RecommendationStatementFinal/breastcancer-screening,
[11] ACOG Practice Bulletin. Clinical management guidelines for obstetrician-gynecologists. Number 42, April 2003. Breast cancer screening. Obstet Gynecol. 2003; 101 (4):821-831.
[12] van Dam P A, Van Goethem M L, Kersschot E, et al. Palpable solid breast masses: retrospective single- and multimodality evaluation of 201 lesions. Radiology. 1988; 166:435-439.
[13] Barton M B, Harris R, Fletcher S W. Does this patient have breast cancer? the screening clinical breast examination: should it be done? how? JAMA. 1999; 282:1270-1280.
[14] Berry D A, Cronin K A, Plevritis S K, et al. Effect of screening and adjuvant therapy on mortality from breast cancer. N Engl J Med. 2005; 353:1784-1792.
[15] Pace L E, Keating N L. A systematic assessment of benefits and risks to guide breast cancer screening decisions. JAMA. 2014; 311:1327-1335.
[16] Kopans D B. Breast Imaging. 2nd ed. Philadelphia, Pa.: Lippincott-Raven Publishers; 1998:409-428.
[17] Moy L, Slanetz P J, Moore R, et al. Specificity of mammography and US in the evaluation of a palpable abnormality: retrospective review. Radiology 2002; 225:176-81.
[18] Gold R H, Bassett L W, Widoff B E, “Highlights from the History of Mammography,” Radiographics 1990; 10:1111-1131.
[19] Otto P M, Blecher C B, “Controversies Surrounding Screening Mammogrraphy,” Mo Med. 2014 September-October; 111(5): 439-443.
[20] Salim M, Dembrower K, Eklund M, Lindholm P, Strand F, “Range of Radiologist Performance in a Population based Screening Cohort of 1 Million Digital Mammography Examinations,” Radiology, 2020 Jul. 28.
[21] Alcusky M, Philpotts L, Bonafede M, Clarke J, Skoufalos A, “The Patient Burden of Screening Mammography Recall,” Journal of Women's Health, 2014 Sep. 23; 23(1).
[22] Oeffinger K C, Fontham E T, Etzioni R, et al. American Cancer Society. Breast Cancer screening for women at average risk: 2015 guideline update from the American Cancer Society. JAMA 2015; 314:1599-1614
[23] Siu A L; U.S. Preventive Services Task Force. Screening for breast cancer: U.S. Preventive Services Task Force recommendation statement. Ann Intern Med 2016; 164:279-296
[24] Monticciolo D L, Newell M S, Hendrick R E, et al. Breast cancer screening for average-risk women: recommendations from the ACR Commission on Breast Imaging. J Am Coll Radiol 2017; 14:1137-1143
[25] American Cancer Society. Cancer Facts & Figures 2019. Atlanta: American Cancer Society; 2019
[26] Breen N et al., “Reported Drop in Mammography: Is This Cause for Concern?” Cancer, 2007 Jun. 14, 109(12): 2405-2409.
[27] Li J J, Shao Z M, “Mammography Screening in Less Developed Countries,” SpringerPlus (2015) 4:615.
[28] Surveillance, Epidemiology, and End Results (SEER) Program (www.seer.cancer.gov) SEER*Stat Database:Incidence—SEER 13 Regs Research Data with Delay-Adjustment, Malignant Only, November 2016 Sub (1992-2014)<Katrina/Rita Population Adjustment>—Linked To County Attributes—Total U.S., 1969-2015 Counties, National Cancer Institute, DCCPS, Surveillance Research Program, released April 2017, based on the November 2016 submission
[29] Consedine N S, Magai C, Krivoshekova Y S, Ryzewicz L, Leugut A I, “Fear, Anxiety, Worry, and Breast Cancer Screening Behavior: A Critical Review,” Cancer Epidemeology, Biomarkers & Prevention, 2004 April, 13(4), 501-510.
[30] Hanson K, Montgomery P, Bakker D, Conlon M, “Factors influencing mammography participation in Canada: an integrative review of the literature,” Curr Oncol. 2009 September; 16(5): 65-75.
[31] Wadden N, Doyle G P, “Breast cancer screening in Canada: A Review,” Can Assoc Radiol J. 2005 December; 56(5):271-5.
[32] Bcheraoui C E et al., “Breast cancer screening in Saudi Arabia: free but almost no takers,” PLoS One, 2015 Mar. 16; 10(3):e0119051. doi: 10.1371/journal.pone.0119051. eCollection 2015.
[33] ACOG Practice Bulletin, “Breast Cancer Risk Assessment and Screening in Average-Risk Women,” July 2017, Bulletin 179, Reaffirmed 2019.
[34] “American Cancer Society Recommendations for the Early Detection of Breast Cancer,” Breast Cancer Early Detection and Diagnosis. https://www.cancer.org/content/dam/CRC/PDF/Public/8579.00.pdf, 5 Mar. 2020.
[35] Hakkinson D J et al., “A significant number of women present with palpable breast cancer even with a normal mammogram within 1 year,” Am J Surg, 2010 December; 200(6): 712-717.
[36] U S Census Data, retrieved from https://www.statista.com/statistics/241488/population-of-the-us-by-sex-and-age/[37] UCSF Radiology Department, https://radiology.ucsf.edu/blog/womens-imaging/mammography-starting-at-age-40-saves-lives
[38] https://www.statista.com/statistics/241488/population-of-the-us-by-sex-and-age/[39] Baguet C R, Commiskey P, Daniel Mullins C, Mishra S I. Recruitment and participation in clinical trials: sociodemographic, rural/urban, and health care access predictors. Cancer Detect Prey. 2006; 30:24-33.
[40] DeSantis C E, Fedewa S A, Goding Sauer A, Kramer J L, Smith R A, Jemal A. Breast cancer statistics, 2015: convergence of incidence rates between black and white women. C A Cancer J Clin. 2016; 66:31-42.
[41] DeSantis C E, Ma J, Goding Sauer A, Newman L A, Jemal A. Breast cancer statistics, 2017, racial disparity in mortality by state. C A Cancer J Clin. 2017; 67:439-448.
[42] Engelman K K, Hawley D B, Gazaway R, Mosier M C, Ahluwalia J S, Ellerbeck E F. Impact of geographic barriers on the utilization of mammograms by older rural women. J Am Geriatr Soc. 2002; 50:62-68.
[43] Siegel R L, Miller K D, Jemal A. Cancer statistics, 2017. C A Cancer J Clin. 2017; 67:7-30.
[44] Wagner M, Anderson K H, Broxton L. Assessment of barriers to screening mammograms for rural, poor, uninsured women and a community plan of action. J Community Health Nurs. 2016; 33:42-53.
[45] Cummings D M, Whetstone L M, Earp J A, Mayne L, “Disparities in mammography screening in rural areas: analysis of county differences in North Carolina,” J Rural Health, Winter 2002; 18(1):77-83.
[46] Blanchard K, Colbert J A, Puri D, et al. Mammographic screening: patterns of use and estimated impact on breast carcinoma survival. Cancer. 2004; 101:495-507.
[47] Michaelson J, Satija S, Moore R, et al. The pattern of breast cancer screening utilization and its consequences. Cancer. 2002; 94:37-43.
[48] Paskett E D, Tatum C, Rushing J, et al. Racial differences in knowledge, attitudes, and cancer screening practices among a triracial rural population. Cancer. 2004; 101:2650-2659.
[49] Peek M E, Han J H. Disparities in screening mammography. Current status, interventions and implications. J Gen Intern Med. 2004; 19:184-194.
[50] Fayanju O M et al., “Perceived Barriers to Mammography among Underserved Women in a Breast Health Center Outreach Program,” Am J Surg. 2014 September; 208(3): 425-434.
[51] Plescia M, White M C, “The National Prevention Strategy and Breast Cancer Screening: Scientific Evidence for Public Health Action,” Am J Public Health. 2013 September; 103(9): 1545-1548.
[52] Trentham-Dietz A, et al. “Tailoring breast cancer screening intervals by breast density and risk for women aged 50 years or older: Collaborative modeling of screening outcomes.” Annals of Internal Medicine. 2016; 165:700.
[53] Miglioretti et al., “Radiation-Induced Breast Cancer Incidence and Mortality from Digital Mammography Screening,” Annals of Internal Medicine, 2016; 164:205-216.
[54] ACOG Statement on Revised American Cancer Society Recommendations on Breast Cancer Screening, Oct. 20, 2015.
[55] Pijpe, A. “Exposure to diagnostic radiation and risk of breast cancer.” BMJ, 2012 www.bmj.com/content/345/bmj.e5660
[56] Breast Cancer Screening: U.S. Preventive Services Task Force. Recommendations; Kirsten Bibbins-Domingo, PhD, M D, Co-Vice Chair, USPSTF, Professor of Medicine and of Epidemiology & Biostatistics University of California, San Francisco.
[57] USPSTF: Final Recommendation Statement: BRCA-Related Cancer: Risk Assessment, Genetic Counseling, and Genetic Testing.
[58] Kaufman C, Son J, Yered E, Sarvazyan A. “Clinical Studies of Palpation Imaging of the Breast on over 1000 patients.” Presented at the 2014 San Antonio Breast Cancer Symposium, Category: Imaging and Screening—Breast Imaging.
[59] IQVIA Institute Report, “Shifts in Healthcare Demand, Delivery, and Care During the COVID-19 Era,” April 2020.

“Preventative Cancer Screenings during COVID-19 Pandemic,” Epic Health Research Network, May 2020.
[61] Skovoroda, A. R., Klishko, A. N., Gusakyan, D. A., Mayevskii, Y. E., Yermilova, V. D., Oranskaya, G. A. and Sarvazyan, A. P. “Quantitative analysis of the mechanical characteristics of pathologically changed soft biological tissues.” Biophysics, 1995; 40(6):1359-64.
[62] Krouskop, T. A., Wheeler, T. M., Kallel, F., Garra, B. S. and Hall, T. “Elastic moduli of breast and prostate tissues under compression.” Ultrason Imaging, 1998; 20(4):260-74.
[63] Sarvazyan, A. P. Elastic properties of soft tissue. In: Handbook of Elastic Properties of Solids, Liquids and Gases, Volume III, Chapter 5, eds. Levy, Bass and Stern, Academic Press. 2001; 107-27.
[64] Parker, K. J., Huang, S. R., Musulin, R. A. and Lerner, R. M. “Tissue response to mechanical vibrations for sonoelasticity imaging.” Ultrasound Med Biol, 1990; 16(3):241-6.
[65] Ophir, J., Cespedes, I., Ponnekanti, H., Yazdi, Y. and Li, X. “Elastography: a quantitative method for imaging the elasticity of biological tissues.” Ultrasonic Imaging, 1991; 13:111-34.
[66] Sarvazyan, A. P., Skovoroda, A. R. et al. Biophysical bases of elasticity imaging. In: Acoustical Imaging, vol. 21, ed. J. P. Jones, Plenum Press, New York and London 1995; 223-40.
[67] Greenleaf, J. F., Fatemi, M. and Insana, M.“Selected methods for imaging elastic properties of biological tissues.” Annu Rev Biomed Eng, 2003; 5:57-78.
[68] Evans, A, Wheelan, P, et al., “Quantitative shear wave ultrasound elastography: initial experience in solid breast masses.” Breast Cancer Research, 2010; 12:R104
[69] Imitiaz, S, “Breast elastography: A New paradigm in diagnostic breast imaging,” Applied Radiology, March 2018; 14-19.
[70] Youk, J, Gweon, H, Son, E. “Shear-wave elastography in breast ultrasonography: the state of the art.” Ultrasonography, 2017 October; 36(4):300-309.
[71] Chang J M, Park I A, Lee S H, Kim W H, Bae M S, Koo H R, Yi A, Kim S J, Cho N, Moon W K. “Stiffness of tumors measured by shear-wave elastography correlated with subtypes of breast cancer.” Eur Radiol, 2013 September; 23(9):2450-8.
[72] Andrew Evans, Patsy Whelehan, Kim Thomson, Denis McLean, Katrin Brauer, Colin Purdie, Lee Jordan, Lee Baker and Alastair Thompson. “Quantitative shear wave ultrasound elastography: initial experience in solid breast masses.” Breast Cancer Research, May 2010
[73] Sarvazyan, A and Egorov, V, “Mechanical Imaging—a Technology for 3-D Visualization and Characterization of Soft Tissue Abnormalities. A Review.” Curr Med Imaging Rev. 2012 Feb. 1; 8(1): 64-73.
[74] Sarvazyan, Egorov, V, et al. “Cost-Effective Screening for Breast Cancer Worldwide: Current State and Future Directions.” Breast Cancer: Basic and Clinical Research, 2008:1 91-99.
[75] Skovoroda, A, Klishko, A, Gusakyan, D, Mayevskii, Y, Yermilova, V, Oranskaya, G, Sarvazyan, A. “Quantitative analysis of the mechanical characteristics of pathologically changed soft biological tissues.” Biophsics, 1995; 40(6):1359-64.
[76] Michaelson, James & Satija, Sameer & Moore, Richard & Weber, Griffin & Halpern, Elkan & Garland, Andrew & Kopans, Daniel & Hughes, Kevin. (2003). Estimates of the Sizes at Which Breast Cancers Become Detectable on Mammographic and Clinical Grounds. Journal of Women's Imaging. 5. 10.1097/00130747-200302000-00002.
[77] Parris, S, Wellman, et al. “Tactile Imaging of Breast Masses, First Clinical Report.” Arch Surg, 2001 February; vol 136.
[78] Kaufman C S, et al., “Objective measurement of the physical exam using a new device: reproducible triage of palpable masses.” Breast Cancer Research and Treatment, 2004; 88; supp 1:5223-4-SABCS
[79] Denis M, et al., “Update on Breast Cancer Detection Using Comb-push Ultrasound Shear Elastography,” IEEE Trans Ultrason Ferroelectr Freq Control. 2015 September; 62(9): 1644-1650.
[80] Nass S J, Henderson I C, Lashof J C, ed., “Ch 5. Evaluation and Cost Coverage of Technologies,” Mammography and Beyond: Developing Techniques for the Early Detection of Breast Cancer. Nat'l Academies Press, 2001.
[81] Fenton J J, Rolnick S J, Harris E L, et al., “Specificity of Clinical Breast Examination in Community Practice,” Journal of General Internal Medicine, March 2007.
[82] Bobo J K, Lee N C, Thames S F, “Findings From 752081 Clinical Breast Examinations Reported to a National Screening Program From 1995 Through 1998,” Journal of the National Cancer Institute, June 2000.
[83] Malmartel, A et. al, “Accuracy of Clinical Breast Examination's Abnormalities for Breast Cancer Screening: Cross-Sectional Study,” European Journal of Obstetrics & Gynecology and Reproductive Biology, April 2019.
[84] Oeffinger K C et al., “Breast Cancer Screening for Women of Average Risk: 2015 Guideline Update from the American Cancer Society,” JAMA, 2015; 314(15):1599-1614.
[85] “Twenty-five-year follow-up for breast cancer incidence and mortality of the Canadian National Breast Screening Study: randomized screening trial.” BMJ; 2014 Feb. 11:348
[86] NIH National Cancer Institute: Mammograms.
[87] Pisano, E. D., Gatsonis, C, Hendrick, E, et al. “Diagnostic performance of digital versus film mammography for breast cancer screening.” N Engl J Med, 2016; 335(17):1773-83.
[88] Nelson, H, O'Meara, E, et al., “Factors Associated with Rates of False-Positive and False Negative Results from Digital Mammography Screening: An Analysis of Registry Data.” Ann Intern Med 2016; 164:226-235.
[89] Lehman, C, et al. “National Performance Benchmarks for Modern Screening Digital Mammography: Update from the Breast Cancer Surveillance Consortium.” Radiology, 2017 April; Volume 283:1.
[90] Miglioretti D L, et al., “When Radiologists Perform Best: The Learning Curve in Screening Mammogram Interpretation,” Radiology. 2009 December; 253(3): 632-640.
[91] Elmore J G et al., “Variability in Interpretive Performance at Screening Mammography and Radiologists' Characteristics Associated with Accuracy,” Radiology. 2009 December; 253(3): 641-651.
[92] Devolli-Disha E, Manxhuka-Kerliu S, Ymeri H, and Kutllovci A, “Comparative Accuracy Of Mammography And Ultrasound In Women With Breast Symptoms According To Age And Breast Density,” Bosn J Basic Med Sci. 2009 May; 9(2): 131-136.
[93] Gonzaga M A, “How accurate is ultrasound in evaluating palpable breast masses?,” Pan Afr Med J. 2010; 7: 1.
[94] Deviolli-Disha E, Manxhuka-Kerliu S, Ymeri H, Kutllovci A, “Comparative accuracy of mammography and ultrasound in women with breast symptoms according to age and breast density,” Bosn J Basic Med Sci. 2009 May; 9(2): 131-136.
[95] Nelson H D, Fu R, Cantor A, Pappas M, Daeges M, Humphrey L. Effectiveness of breast cancer screening: systematic review and meta-analysis to update the 2009 U.S. Preventive Services Task Force Recommendation. Ann Intern Med. 164(4):244-55, 2016.
[96] Yankaskas B C, Cleveland R J, et al, Association of Recall Rates with Sensitivity and Positive Predictive Values of Screening Mammography AJR 2001; 177:543-549.
[97] DeNeef P, Gandara J: Experience with indeterminate mammograms. West J Med 1991 January; 154:36-39
[98] Throckmorton A D, et al. Dense breasts: What do our patients need to be told and why? Annals of Surgical Oncology. 2016; 23:3119.
[99] Nelson H D, Tyne K, Naik A, et al. Screening for breast cancer: an update for the U.S. Preventive Services Task Force. Ann Intern Med 2009; 151:727-73.
[100] Kaufman C S et al., Digital documentation of the physical examination: moving the clinical breast exam to the electronic medical record, Am J Surg, 192(2006) 444-449.

1. A method comprising:
receiving, by a processor, at a data collection appliance and gateway, a first data set associated with a pressure elastography measurement of a breast mass detection procedure conducted on a subject;
receiving, by the processor, at the data collection appliance and gateway, a second data set associated with an ultrasound scan of a second breast mass detection procedure contemporaneously conducted on the subject following the pressure elastography measurement;
storing, by the processor, at the data collection appliance and gateway, the first data set and the second data set; and
transmitting, by the processor of the data collection appliance and gateway, the first data set and the second data set to a local and/or remote cloud server,
wherein the first data set and the second data set are (i) subsequently presented on a display of a computing device or in a report for use in an assessment of a breast mass and/or (ii) subsequently analyzed via machine learning or deep learning operations to provide an indication associated with the assessment of the breast mass.

2. The method of claim 1 further comprising:
analyzing, by a processor of the local and/or remote cloud server or the data collection appliance and gateway, the first data set to assess metrics associated with quality of acquisition of the pressure elastography measurement.

3. The method of claim 2, wherein the metrics associated with quality of acquisition comprises at least one of:
metrics associated with examiner monitoring;
metrics associated with workflow monitoring; and
metrics associated with device monitoring.

4. The method of claim 3, wherein the metrics associated with examiner monitoring comprise any one of:
a log of time spent per breast during an exam;
a log of percent time outside pre-defined force region;
a log of a number of recordings deleted;
a log of a number of re-recording;
a log of total time spent per breast and per patient during an exam;
a log of an average force used during a different portion of the exam;
a log of variations from recommendations and deviations from pre-defined ranges of operations.

5. The method of claim 3, wherein the metrics associated with workflow monitoring comprise a log of significant deviations from the training may indicate a need to change recommendations.

6. The method of claim 3, wherein the metrics associated with device monitoring comprises at least one of:
hardware status;
a log of hardware status indicative of wear; and
a log of daily calibration data;
a log of defects associated with a manufactured lot or manufacturer.

7. The method of claim 1 further comprising:
acquiring, by the processor, a third data set associated with the subject, the breast mass detection procedure, and the second breast mass detection procedure; and
transmitting, by the processor, the third data set to the local and/or remote cloud server, wherein the third data set are used with the first data set and the second data set to be (i) subsequently presented on the display of the computing device or in the report for use in the assessment of the breast mass or (ii) subsequently analyzed via the machine learning or deep learning operations to provide the indication associated with the assessment of the breast mass.

8. The method of claim 1, wherein the first data set is acquired by:
authenticating, by a processor, an examiner credential comprising an examiner identifier;
retrieving, by the processor, from a local and/or remote cloud database, through the data collection appliance and gateway, a data set comprising a list of clinic or center; and
presenting, by the processor, at a user interface, the list of clinic or center, wherein the examiner identifier is associated with at least the pressure elastography measurement and used for quality monitoring associated with the examiner identifier.

9. The method of claim 2 further comprising:
presenting, through a teleradiology interface, focused ultrasounds of detected masses while exam in-process.

10. The method of claim 1, further comprising:
transmitting, by the processor, a notification of a potential mass to an on-call radiologists for review upon the identification of the mass by an automated analysis system.

11. The method of claim 1, further comprising:
generating, by processor, a report of the pressure elastography measurement; and
transmitting, by the processor, the report to a radiologist or pre-defined reviewer for review.

12.-17. (canceled)

18. A method of claim 1 comprising:
determining, by the processor, an estimated size and estimated relative hardness of a detected mass; and
transmitting, by the processor, the first data set and estimated size and estimated relative hardness of the detected mass to a local and/or remote cloud server, wherein the first data set is (i) subsequently presented on a display of a computing device or in a report for use in an assessment of a breast mass or (ii) subsequently analyzed via machine learning or deep learning operations to provide an indication associated with the assessment of the breast mass.

19.-25. (canceled)

26. The method of claim 18, wherein the first data set is used by the machine learning or deep learning operation to output a classification output value selected from the group consisting of:
a classification code or identifier associated with no mass detected;
a classification code or identifier associated a pre-existing, known-benign mass;
a classification code or identifier associated with a new mass;
a classification code or identifier associated with a known, confirmed cancer.

27. A system comprising:
a processor; and
a memory having instructions stored thereon, wherein execution of the instructions by the processor causes the processor to:
acquire, via an acquisition device comprising a capacitive sensor array, a first data set associated with a pressure elastography measurement of a breast mass detection procedure conducted on a subject;
determine, an estimated size and estimated relative hardness of a detected mass; and
transmit, the first data set and estimated size and estimated relative hardness of the detected mass to a local and/or remote cloud server, wherein the first data set is (i) subsequently presented on a display of a computing device or in a report for use in an assessment of a breast mass or (ii) subsequently analyzed via machine learning or deep learning operations to provide an indication associated with the assessment of the breast mass,
wherein the system comprises distributed local controller configured to keep a local cache of data relevant only to a given collection center for a pre-defined time duration or scans, wherein the acquisition devices can acquire and operate without network connectivity or connection to a central database for the pre-defined time duration or scans

28. The system of claim 27, comprising a cellular and/or Wifi-network interface.

29. The system of claim 27 further comprises at least one of a router, VPN, firewall, gateway, and bridge.

30. The system of claim 27 further comprising a Web server and/or a DICOM server.

31. An enterprise software system comprising a cloud-based clinical database and processing, the enterprise software system having instructions stored thereon, wherein execution of the instructions by a processor causes the processor to:
acquire, via an acquisition device comprising a capacitive sensor array, a first data set associated with a pressure elastography measurement of a breast mass detection procedure conducted on a subject;
determine, an estimated size and estimated relative hardness of a detected mass; and
transmit, the first data set and estimated size and estimated relative hardness of the detected mass to a local and/or remote cloud server, wherein the first data set is (i) subsequently presented on a display of a computing device or in a report for use in an assessment of a breast mass or (ii) subsequently analyzed via machine learning or deep learning operations to provide an indication associated with the assessment of the breast mass.

32. The enterprise software system of claim 31 further comprising:
an examiner quality monitoring module.

33. The enterprise software system of claim 31 further comprising one or more modules selected from the group consisting of:
a teleradiology interface module;
a remote examiner training module;
an exportable electronic record module;
a device integrity monitoring module;
a management & billing information module; and
a device/fleet firmware and software update module.
